(19) United States
(12) Patent Application Publication
(10) Pub. No.: US 2024/0216239 A1
(43) Pub. Date: Jul. 4, 2024

(54) LIQUID OILY COMPOSITION AND COSMETIC CONTAINING SAME

(71) Applicant: The Nisshin OilliO Group, Ltd., Tokyo (JP)

(72) Inventors: Hisanori Kachi, Yokohama-shi (JP); Keiichi Oyama, Yokohama-shi (JP)

(21) Appl. No.: 18/588,758

(22) Filed: Feb. 27, 2024

Related U.S. Application Data
(63) Continuation of application No. PCT/JP2022/033105, filed on Sep. 2, 2022.

(30) Foreign Application Priority Data
Sep. 9, 2021 (JP) 2021-146641

(51) Int. Cl. A61K 8/37; A61K 8/06; A61K 8/39; A61K 8/891; A61Q 1/02; A61Q 17/04; A61Q 19/00

(52) U.S. Cl. CPC A61K 8/37; A61K 8/062; A61K 8/064; A61K 8/39; A61K 8/891; A61Q 1/02; A61Q 17/04; A61Q 19/00

(57) ABSTRACT

The present invention relates to a liquid oily composition and the like, the liquid oily composition containing: a component (A) which is an ester oil having a 2-(1,3,3-trimethylbutyl)-5,7,7-trimethyloctyl structure in the aliphatic chain, having a hydroxyl value within a range from 5 to 100 mgKOH/g, and being liquid at 20° C.; and a component (B) which is a silicone oil that is miscible with the component (A) in a mass ratio of 1:1 and is liquid at 20° C., wherein the mass ratio ((A)/(B)) of the amount of the component (A) with respect to the total composition relative to the amount of the component (B) with respect to the total composition is within a range from 0.05 to 4.

### TECHNICAL FIELD

The present invention relates to a liquid oily composition which has a light feeling, an excellent water repellency effect and excellent pigment dispersibility, and is capable of imparting persistence to a cosmetic film when used as a base oil in any of various cosmetics, and also relates to cosmetics containing the liquid oily composition. Priority is claimed on Japanese Patent Application No. 2021-146641, filed Sep. 9, 2021, the content of which is incorporated herein by reference.

### BACKGROUND ART

Cosmetics composed of oils, water, and powders and the like require favorable ease of application to the skin, beauty of the resulting cosmetic film, or persistence of the cosmetic film, and oils having various characteristics are used as components for controlling these properties. However, for example, although oils of low viscosity have a light feeling and can be easily applied to the skin, the persistence of the cosmetic film tends to be inferior. In contrast, oils of high viscosity are difficult to spread, and can sometimes result in increased feelings of stickiness.

Known components that have a light feeling and good persistence of the cosmetic film due to high water repellency include various silicone compounds such as silicone oils and silicone resins. However, silicone compounds have a number of drawbacks, including poor compatibility with other cosmetic raw materials and poor pigment dispersibility, which can sometimes lead to separation or pigment aggregation over time, causing problems such as a lack of uniformity in the resulting cosmetic film or poor storage stability.

In terms of improving the persistence of the cosmetic film, the use of various silicone compounds has been proposed. For example, Patent Document 1 discloses a cosmetic in which, by creating a coating film using a film-forming silicone resin and employing technology that prevents cosmetic deterioration caused by mixing of the cosmetic film with sebum, a cosmetic is obtained that exhibits excellent cosmetic persistence, a natural glossy feeling, smooth spreadability, and excellent stability over time. Further, Patent Document 2 discloses an emulsion cosmetic in which, by adding a specific powder in addition to a film-forming silicone resin, effects are obtained that include favorable spreading, smoothness, low impact on the skin, good persistence of the cosmetic effect over long periods, and excellent uniformity of the cosmetic film. However, although these film-forming silicone resins exhibit light spreadability and excellent persistence of the cosmetic film, they do not adequately resolve the issue of compatibility with other components, and therefore care must be taken regarding compatibility with the other blend components in order to achieve satisfactory storage stability of the formulation, and the pigment dispersibility is not entirely satisfactory. Further, Patent Document 3 discloses that a monoester obtained by esterifying a branched fatty acid and a branched alcohol and used for improving the compatibility with silicone compounds improves the sensations upon use such as the skin compatibility and the feeling of body, but this compound is also not entirely satisfactory in terms of the water repellency effect and pigment dispersibility.

### CITATION LIST

### Patent Documents

### [Patent Document 1]

Japanese Unexamined Patent Application, First Publication No. 2002-47129

### [Patent Document 2]

Japanese Unexamined Patent Application, First Publication No. 2006-265213

### [Patent Document 3]

Japanese Unexamined Patent Application, First Publication No. 2017-8000

### SUMMARY OF INVENTION

### Technical Problem

Against this type of background, technology that provides a light feeling, an excellent water repellency effect or excellent pigment dispersibility, is capable of imparting persistence to a cosmetic film and excellent storage stability when used as a base oil in any of various cosmetics, exhibits favorable coloring properties and minimal dulling after application to the skin, or is capable of imparting water resistance would be very desirable.

An object of the present invention is to provide a liquid oily composition that has a light feeling, an excellent water repellency effect and excellent pigment dispersibility, is capable of imparting persistence to a cosmetic film and excellent storage stability when used as a base oil in any of various cosmetics, exhibits favorable coloring properties and minimal dulling after application to the skin, or is capable of imparting water resistance, and also to provide cosmetics containing the liquid oily composition.

### Solution to Problem

Following intensive investigation, the inventors of the present invention discovered that a liquid oily composition prepared by mixing, in a specific mass ratio, a liquid ester compound having a 2-(1,3,3-trimethylbutyl)-5,7,7-trimethyloctyl structure in the aliphatic chain and having a hydroxyl value within a specific range, and a silicone oil capable of being mixed uniformly and transparently with the liquid ester compound in a mass ratio of 1:1 and being liquid at 20° C., exhibited a light feeling, had an excellent water repellency effect or excellent pigment dispersibility, was capable of imparting excellent persistence to the cosmetic film or excellent storage stability when used as a base oil in any of various cosmetics, exhibited favorable coloring properties, yielded minimal dulling after application to the skin or was capable of imparting water resistance, enabling them to complete the present invention.

In other words, a liquid oily composition and cosmetic according to the present invention includes the following aspects [1] to [10].
[1] A liquid oily composition containing a component (A) and a component (B) described below, wherein
   the mass ratio ((A)/(B)) of the amount of the component (A) with respect to the total composition, relative to the amount of the component (B) with respect to the total composition, is within a range from 0.05 to 4.
   Component (A): an ester oil having a 2-(1,3,3-trimethylbutyl)-5,7,7-trimethyloctyl structure in the aliphatic chain, having a hydroxyl value within a range from 5 to 100 mgKOH/g, and being liquid at 20° C.
   Component (B): a silicone oil that is miscible with the component (A) in a mass ratio of 1:1 and is liquid at 20° C.
[2] The liquid oily composition according to [1], wherein the component (A) is at least one compound selected from the group consisting of polyglyceryl-2 tri(2-(1,3,3-trimethylbutyl)-5,7,7-trimethyloctanoate) and di(2-(1,3,3-trimethylbutyl)-5,7,7-trimethyloctyl) malate.
[3] The liquid oily composition according to [1] or [2], wherein the component (B) is at least one compound selected from the group consisting of dimethicone, diphenylsiloxy phenyl trimethicone, diphenyl dimethicone and phenyl trimethicone.
[4] The liquid oily composition according to any one of [1] to [3], wherein the component (A) is polyglyceryl-2 tri(2-(1,3,3-trimethylbutyl)-5,7,7-trimethyloctanoate), and the component (B) is dimethicone.
[5] The liquid oily composition according to any one of [1] to [4], wherein the component (B) has a viscosity at 20° ° C. within a range from 2 to 300 mPa·s.
[6] A cosmetic containing the liquid oily composition according to any one of [1] to [5].
[7] A cosmetic containing a component (A) and a component (B) described below, wherein
   the mass ratio ((A)/(B)) of the amount of the component (A) with respect to the total mass of the cosmetic, relative to the amount of the component (B) with respect to the total mass of the cosmetic, is within a range from 0.05 to 4.
   Component (A): an ester oil having a 2-(1,3,3-trimethylbutyl)-5,7,7-trimethyloctyl structure in the aliphatic chain, having a hydroxyl value within a range from 5 to 100 mgKOH/g, and being liquid at 20° ° C.
   Component (B): a silicone oil that is miscible with the component (A) in a mass ratio of 1:1 and is liquid at 20° C.
[8] The cosmetic according to [6] or [7], further containing a component (C) described below.
   Component (C): a powder.
[9] The cosmetic according to any one of [6] to [8], further containing a component (D) described below.
   Component (D): a volatile oil that is a volatile hydrocarbon and/or a volatile silicone.
[10] The cosmetic according to any one of [6] to [9], further containing a component (E) and a component (F) described below, and being an O/W emulsion cosmetic or a W/O emulsion cosmetic.
   Component (E): water.
   Component (F): a surfactant
[11] A water resistance-imparting agent containing the liquid oily composition according to any one of [1] to [5] as an active component.

### Effects of Invention

The liquid oily composition according to the present invention has a light feeling, an excellent water repellency effect or excellent pigment dispersibility, and is capable of imparting persistence to a cosmetic film or excellent storage stability when used as a base oil in any of various cosmetics. As a result, the liquid oily composition is particularly suitable as a raw material for makeup cosmetics and sunscreen cosmetics and the like that require favorable persistence of the cosmetic film.

Further, by including the above liquid oily composition as one of the oily components, a cosmetic according to the present invention exhibits favorable sensations upon use, with excellent persistence of the cosmetic film, favorable coloring properties and minimal dulling, or exhibiting excellent storage stability or enabling the imparting of water resistance.

### DESCRIPTION OF EMBODIMENTS

### <Liquid Oily Composition>

The liquid oily composition according to the present invention contains, as a component (A), an ester oil having a 2-(1,3,3-trimethylbutyl)-5,7,7-trimethyloctyl structure in the aliphatic chain, having a hydroxyl value within a range from 5 to 100 mgKOH/g, and being liquid at 20° C., and also contains, as a component (B), a silicone oil that is miscible with the component (A) in a mass ratio of 1:1 and is liquid at 20° C. Silicone oils are already widely known as conventional water repellent materials. In the present invention, by mixing the silicone oil with the specified liquid ester oil in a specific mass ratio, the water repellency effect is enhanced, and the respective drawbacks of the liquid ester oil and the silicone oil are offset.

The component (A) is described below in further detail. The ester oil having a 2-(1,3,3-trimethylbutyl)-5,7,7-trimethyloctyl structure in the aliphatic chain, having a hydroxyl value within a range from 5 to 100 mgKOH/g, and being liquid at 20° C. describes an ester oil which is a compound having an ester structure (also referred to as an esterified material), in which at least one of the carboxylic acid-derived structural portion and the alcohol-derived structural portion has a 2-(1,3,3-trimethylbutyl)-5,7,7-trimethyloctyl structure, and which has a hydroxyl value within a range from 5 to 100 mgKOH/g and is liquid at 20° C., and more specifically, refers to either an esterified material obtained by an esterification reaction between 2-(1,3,3-trimethylbutyl)-5,7,7-trimethyloctanoic acid and a polyol having two or more hydroxyl groups within the molecule, or an esterified material obtained by an esterification reaction between 2-(1,3,3-trimethylbutyl)-5,7,7-trimethyloctanol and a hydroxy acid having at least one carboxylic acid group and at least one hydroxyl group within the molecule.

Both 2-(1,3,3-trimethylbutyl)-5,7,7-trimethyloctanoic acid and 2-(1,3,3-trimethylbutyl)-5,7,7-trimethyloctanol are conventionally known compounds, and these compounds may be either produced in accordance with conventional methods, or commercially available products may be used.

Examples of the polyol having two or more hydroxyl groups within the molecule that is subjected to the esterification reaction with 2-(1,3,3-trimethylbutyl)-5,7,7-trimethyloctanoic acid include 1,2-propanediol, 1,3-propanediol, 1,3-butylene glycol, neopentyl glycol, dipropylene glycol, glycerol, diglycerol, triglycerol, tetraglycerol, pentaerythritol and dipentaerythritol, and among these compounds, glycerol and diglycerol are preferred.

Examples of the organic acid having at least one carboxylic acid group and at least one hydroxyl group within the molecule that is subjected to the esterification reaction with 2-(1,3,3-trimethylbutyl)-5,7,7-trimethyloctanol include lactic acid, malic acid, citric acid, glyceric acid and tartaric acid, and among these compounds, malic acid is preferred.

The liquid ester oil of the component (A) has a hydroxyl value within a range from 5 to 100 mgKOH/g.

In this description, the “hydroxyl value” represents the number of milligrams of potassium hydroxide needed to neutralize the amount of acetic acid required to acetylate all the hydroxyl groups within a 1 g sample, and can be determined, for example, by esterifying the sample using a pyridine solution of acetic anhydride, and then titrating the unreacted excess acetic acid with a potassium hydroxide solution.

A liquid ester oil with a hydroxyl value that falls within the above range can be obtained by conducting the esterification reaction with the molar ratio between the 2-(1,3,3-trimethylbutyl)-5,7,7-trimethyloctanoic caid and the polyol adjusted so that hydroxyl groups remain following the esterification reaction. Further, in those cases where 2-(1,3,3-trimethylbutyl)-5,7,7-trimethyloctanol is subjected to an esterification reaction with a hydroxy acid having at least one carboxylic acid group and at least one hydroxyl group within the molecule, by adjusting the molar ratio between the two reactants in a similar manner, a liquid ester oil having the targeted hydroxyl value can be obtained.

The hydroxyl value of the component (A) is within a range from 5 to 100 mgKOH/g, and is preferably within a range from 10 to 90 mgKOH/g, more preferably from 20 to 80 mgKOH/g, and most preferably from 30 to 60 mgKOH/g. When the hydroxyl value of the component (A) falls within the above range, the water repellency and pigment dispersibility effects of the present invention are particularly superior. If the hydroxyl value of the component (A) is less than 5 mgKOH/g, then the pigment dispersibility of the liquid oily composition may sometimes deteriorate. If the hydroxyl value of the component (A) is greater than 100 mgKOH/g, then the feel of the liquid oily composition becomes heavy, and the water repellency may sometimes deteriorate.

The ester oil of the component (A) is preferably at least one compound selected from the group consisting of:
glycerol esters such as glyceryl di(2-(1,3,3-trimethylbutyl)-5,7,7-trimethyloctanoate) and glyceryl tri(2-(1,3,3-trimethylbutyl)-5,7,7-trimethyloctanoate);
polyglycerol-2 esters such as polyglyceryl-2 tri(2-(1,3,3-trimethylbutyl)-5,7,7-trimethyloctanoate) and polyglyceryl-2 tetra(2-(1,3,3-trimethylbutyl)-5,7,7-trimethyloctanoate);
polyglycerol-3 esters such as polyglyceryl-3 tri(2-(1,3,3-trimethylbutyl)-5,7,7-trimethyloctanoate), polyglyceryl-3 tetra(2-(1,3,3-trimethylbutyl)-5,7,7-trimethyloctanoate), and polyglyceryl-3 penta(2-(1,3,3-trimethylbutyl)-5,7,7-trimethyloctanoate);
polyglycerol-4 esters such as polyglyceryl-4 tetra(2-(1,3,3-trimethylbutyl)-5,7,7-trimethyloctanoate), polyglyceryl-4 penta(2-(1,3,3-trimethylbutyl)-5,7,7-trimethyloctanoate), and polyglyceryl-4 hexa(2-(1,3,3-trimethylbutyl)-5,7,7-trimethyloctanoate);
polyglycerol-5 esters such as polyglyceryl-5 tetra(2-(1,3,3-trimethylbutyl)-5,7,7-trimethyloctanoate), polyglyceryl-5 penta(2-(1,3,3-trimethylbutyl)-5,7,7-trimethyloctanoate), polyglyceryl-5 hexa(2-(1,3,3-trimethylbutyl)-5,7,7-trimethyloctanoate), and polyglyceryl-5 hepta(2-(1,3,3-trimethylbutyl)-5,7,7-trimethyloctanoate);
polyglycerol-6 esters such as polyglyceryl-6 penta(2-(1,3,3-trimethylbutyl)-5,7,7-trimethyloctanoate), polyglyceryl-6 hexa(2-(1,3,3-trimethylbutyl)-5,7,7-trimethyloctanoate), polyglyceryl-6 hepta(2-(1,3,3-trimethylbutyl)-5,7,7-trimethyloctanoate), and polyglyceryl-6 octa(2-(1,3,3-trimethylbutyl)-5,7,7-trimethyloctanoate);
polyglycerol-10 esters such as polyglyceryl-10 hepta(2-(1,3,3-trimethylbutyl)-5,7,7-trimethyloctanoate), polyglyceryl-10 octa(2-(1,3,3-trimethylbutyl)-5,7,7-trimethyloctanoate), polyglyceryl-10 nona(2-(1,3,3-trimethylbutyl)-5,7,7-trimethyloctanoate), polyglyceryl-10 deca(2-(1,3,3-trimethylbutyl)-5,7,7-trimethyloctanoate), polyglyceryl-10 undeca(2-(1,3,3-trimethylbutyl)-5,7,7-trimethyloctanoate), and polyglyceryl-10 dodeca(2-(1,3,3-trimethylbutyl)-5,7,7-trimethyloctanoate);
malate esters such as di(2-(1,3,3-trimethylbutyl)-5,7,7-trimethyloctyl) malate;
pentaerythritol esters such as pentaerythrityl tri(2-(1,3,3-trimethylbutyl)-5,7,7-trimethyloctanoate) and pentaerythrityl tetra(2-(1,3,3-trimethylbutyl)-5,7,7-trimethyloctanoate); and
dipentaerythritol esters such as dipentaerythrityl tetra(2-(1,3,3-trimethylbutyl)-5,7,7-trimethyloctanoate), dipentaerythrityl penta(2-(1,3,3-trimethylbutyl)-5,7,7-trimethyloctanoate), and dipentaerythrityl hexa(2-(1,3,3-trimethylbutyl)-5,7,7-trimethyloctanoate).

The ester oil of the component (A) included in the liquid oily composition according to the present invention is preferably at least one compound selected from the group consisting of polyglyceryl-2 tri(2-(1,3,3-trimethylbutyl)-5,7,7-trimethyloctanoate), glyceryl tri(2-(1,3,3-trimethylbutyl)-5,7,7-trimethyloctanoate) and di(2-(1,3,3-trimethylbutyl)-5,7,7-trimethyloctyl) malate, is more preferably at least one compound selected from the group consisting of polyglyceryl-2 tri(2-(1,3,3-trimethylbutyl)-5,7,7-trimethyloctanoate) and di(2-(1,3,3-trimethylbutyl)-5,7,7-trimethyloctyl) malate, and is most preferably polyglyceryl-2 tri(2-(1,3,3-trimethylbutyl)-5,7,7-trimethyloctanoate).

The ester oil of the component (A) can be produced using conventionally known methods, or commercially available products may be used. Examples of commercially available products that can be used favorably include COSMOL 43N (representative hydroxyl value: 40 mgKOH/g), SALACOS 3318 (representative hydroxyl value: 20 mgKOH/g), COSMOL 222 (representative hydroxyl value: 80 mgKOH/g), and SALACOS DP-518N (representative hydroxyl value: 30 mgKOH/g) (all manufactured by The Nisshin OilliO Group, Ltd. Among these products, at least one compound selected from the group consisting of COSMOL 43N, SALACOS 3318 and COSMOL 222 is preferred, at least one compound selected from the group consisting of COSMOL 43N and COSMOL 222 is more preferred, and COSMOL 43N is particularly desirable.

Next is a more detailed description of the component (B). The term “silicone oil” is a generic term for liquid substances having a structure represented by general formula (1) shown below (wherein in formula (1), n represents the degree of polymerization, which is typically an integer of 3 or greater, and the plurality of R groups may be the same or different, with each representing a methyl group or a phenyl group), and the component (B) used in the present invention is a compound in which the structure of each R group is a methyl group or a phenyl group. Compounds typically known as dimethicone have a methyl group bonded at each of the R positions, and increase in viscosity as the degree of polymerization increases. Accordingly, each dimethicone is typically distinguished using a viscosity value.

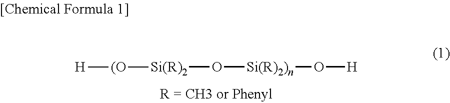

Further, the expression “miscible with the component (A) in a mass ratio of 1:1” indicates that when a silicone oil of the same mass as that of the ester oil of the component (A) is weighed into a container, the two components are heated and stirred at 80° C. to form a uniform liquid, and the resulting mixture is then left to stand at 20° C., no visual separation or cloudiness occurs, and a transparent state is maintained.

Any silicone oil that is miscible with the component (A) in a mass ratio of 1:1 and is liquid at 20° C. can be used as the component (B) used in the present invention. A single oil may be used alone as the silicone oil of the component (B), or a combination of two or more silicone oils may be used. Among the various possibilities, preferred oils for the component (B) include at least one compound selected from the group consisting of dimethicone, diphenylsiloxy phenyl trimethicone, diphenyl dimethicone and phenyl trimethicone, particularly preferred compounds include at least one compound selected from the group consisting of dimethicone 5 cs, dimethicone 10 cs, dimethicone 30 cs, diphenylsiloxy phenyl trimethicone and diphenyl dimethicone, and even more preferred compounds include at least one compound selected from the group consisting of dimethicone 5 cs, dimethicone 10 cs, diphenylsiloxy phenyl trimethicone and diphenyl dimethicone. The units “cs” indicate the viscosity represented as CGS units, whereas the “mPa·s” units used below indicate the viscosity represented as SI units, wherein 1 cs=1 mPa·s. By using a silicone oil that is miscible with the component (A) in a mass ratio of 1:1 and is liquid at 20° C. as the component (B), excellent water repellency and pigment dispersibility are achieved. If a silicone oil that is not miscible with the component (A) in a mass ratio of 1:1 is used instead of the component (B), then the water repellency effect is weaker, and the pigment dispersibility effect also weakens.

The silicone oil used as the component (B) in the present invention preferably has a viscosity at 20° C. that is within a range from 2 to 1,000 mPa·s, more preferably from 5 to 500 mPa·s, and most preferably from 10 to 300 mPa·s. Provided the viscosity at 20° C. of the silicone oil of the component (B) falls within the above range, the effects of the present invention of a light feeling and an excellent water repellency effect manifest strongly. If the viscosity at 20° C. of the silicone oil of the component (B) is less than 2 mPa·s, then the water repellency effect may sometimes weaken, whereas if the viscosity exceeds 1,000 mPa·s, the feeling of the composition may become heavier.

In one aspect, the silicone oil used as the component (B) has a viscosity at 20° C. within a range from 3 to 20 mPa·s, whereas in another aspect, the viscosity is within a range from 5 to 10 mPa·s. Further, in another aspect, the silicone oil used as the component (B) is a dimethicone with a viscosity at 20° ° C. within a range from 3 to 20 mPa·s, and in yet another aspect, the silicone oil used as the component (B) is a dimethicone with a viscosity at 20° ° C. within a range from 5 to 10 mPa·s.

In the present description, the “viscosity” can be measured using a B-type viscometer.

In the liquid oily composition according to the present invention, the mass ratio of the amount of the component (A) within the composition relative to the amount of the component (B) within the composition ([amount (mass) of the component (A)]/[amount (mass) of the component (B)]) (hereinafter sometimes referred to as the “(A)/(B) ratio”) is within a range from 0.05 to 4. The (A)/(B) ratio of the liquid oily composition according to the present invention is preferably within a range from 0.10 to 1.5, more preferably from 0.17 to 0.7, even more preferably from 0.25 to 0.45, and most preferably from 0.30 to 0.40. By ensuring that the (A)/(B) ratio falls within the above range, the liquid oily composition exhibits a light feeling, has high water repellency and improved pigment dispersibility, and when the liquid oily composition is used as one of the raw materials in a cosmetic, the persistence of the cosmetic film improves, and the storage stability is also favorable, both of which are desirable. In contrast, in liquid oily compositions in which the amount of the component (A) is small and the mass ratio falls outside the above range, the water repellency effect is weaker, and the pigment dispersibility may also sometimes weaken. Further, in liquid oily compositions in which the amount of the component (A) is large and the mass ratio falls outside the above range, the water repellency effect is weaker, and the feeling of the composition may become heavier.

The liquid oily composition according to the present invention may be a composition composed only of the component (A) and the component (B), or may also contain one or more other components in addition to the component (A) and the component (B). In those cases where the composition contains other components, the proportion of the combined mass of the component (A) and the component (B), relative to the total mass of the liquid oily composition according to the present invention, is preferably at least 80% by mass but less than 100% by mass, and more preferably at least 90% by mass but less than 100% by mass. The other components may include the types of additives typically added to external skin compositions such as cosmetics (in this description, sometimes referred to as “additives permitted as cosmetics”), provided the effects of the present invention are not impaired.

The expression “additives permitted as cosmetics” refers to additives typically added to cosmetics, and means additives which exhibit no toxicity and are physiologically permitted in the types of amounts typically added in the field of cosmetics. Examples of these types of additives include the types of components described below in the section relating to “cosmetics”, including oily components (but excluding the component (A) and the component (B)), antioxidants, antioxidant assistants, preservatives, ultraviolet absorbers, monohydric alcohols, polyhydric alcohols, water-soluble polymers, pH regulators, inorganic salts or salts of organic acids, chelating agents, vitamins, organic solvents, fragrances, various extracts, and ultraviolet scattering agents. Specific examples include the additives mentioned below.

The amount of these additives permitted as cosmetics, relative to the total mass of the liquid oily composition, may be within a range from 0 to 20% by mass, and preferably from 0 to 10% by mass.

The liquid oily composition can be produced by mixing the component (A), the component (B), and any additives permitted as cosmetics that may be included according to need. The mixing may be conducted by placing all the components in a mixing device simultaneously, or by conducting mixing while each component is added in an appropriate sequence. The stirring or kneading conducted during the mixing may use methods well known in the technical field. The temperature during the mixing may be set so that the temperature inside the mixer is within a range from 20 to 100° C.

The liquid oily composition may be used, for example, by addition to a cosmetic described below as a production raw material for the cosmetic. In other words, one aspect of the present invention relates to use of the liquid oily composition for the production of a cosmetic, and another aspect relates to use of the liquid oily composition as a cosmetic.

### <Cosmetic>

A cosmetic according to the present invention contains the component (A) and the component (B) in amounts such that the (A)/(B) ratio falls within a range from 0.05 to 4, and also contains one or more components typically added to cosmetic products (in this description, sometimes referred to as “additives permitted as cosmetics”). In other words, one embodiment of the cosmetic of the present invention contains the component (A), the component (B), and one or more additives permitted as cosmetics, wherein the component (A) and the component (B) are included in amounts such that the (A)/(B) ratio falls within a range from 0.05 to 4. Here, the “additives permitted as cosmetics” may have the same definition as the “additives permitted as cosmetics” described above in the section relating to the liquid oily composition.

One embodiment of the cosmetic of the present invention contains a combined mass of the component (A) and the component (B), relative to the total mass of the cosmetic, that is at least 40% by mass but less than 100% by mass, and contains one or more additives permitted as cosmetics in an amount exceeding 0% by mass but not more than 60% by mass. Another embodiment of the cosmetic of the present invention contains a combined mass of the component (A) and the component (B), relative to the total mass of the cosmetic, that is at least 60% by mass but less than 100% by mass, and contains one or more additives permitted as cosmetics in an amount exceeding 0% by mass but not more than 40% by mass. Yet another embodiment of the cosmetic of the present invention contains a combined mass of the component (A) and the component (B), relative to the total mass of the cosmetic, that is at least 80% by mass but less than 100% by mass, and contains one or more additives permitted as cosmetics in an amount exceeding 0% by mass but not more than 20% by mass. Yet another embodiment of the cosmetic of the present invention contains a combined mass of the component (A) and the component (B), relative to the total mass of the cosmetic, that is at least 90% by mass but less than 100% by mass, and contains one or more additives permitted as cosmetics in an amount exceeding 0% by mass but not more than 10% by mass. Here, the sum of the amounts of the component (A), the component (B) and the additives permitted as cosmetics must not exceed 100% by mass but may be 100% by mass.

There are no particular limitations on the external appearance, the form, or the fluidity of the cosmetic. The cosmetic may have an external appearance that is transparent, translucent, or emulsified (cloudy), and may have a form that is liquid, cream-like, or solid. Preferred examples of the cosmetic according to the present invention include makeup cosmetics such as powder foundations, water-in-oil emulsion foundations, sunscreens, eye shadows, rouges and lipsticks, as well as skincare cosmetics such as milky lotions, creams, essences and lotions.

The cosmetic according to the present invention preferably also contains, in addition to the component (A) and the component (B), a powder as a component (C). Further, the component (C) may also be added instead of an additive permitted as a cosmetic. In other words, one aspect of the cosmetic of the present invention contains the component (A), the component (B), the component (C), and one or more additives permitted as cosmetics, where another aspect contains the component (A), the component (B), and the component (C).

In those cases where the cosmetic according to the present invention is a powder cosmetic containing the component (C), the amount of the liquid oily composition according to present invention within the powder cosmetic (namely, the proportion of the combined amounts of the component (A) and the component (B)), relative to the total mass of the powder cosmetic, is preferably within a range from 0.1 to 30% by mass. The amount of the powder within the cosmetic preferably represents 2 to 98% by mass of the total cosmetic. The amount of the powder within the cosmetic may be adjusted to a more appropriate level depending on the type of cosmetic, and for example, in the case of a powder foundation, rouge or eye shadow, an amount of 70 to 98% by mass of the total cosmetic is ideal, in the case of a water-in-oil emulsion foundation, emulsion-type eye shadow or sunscreen, an amount of 5 to 30% by mass of the total cosmetic is ideal, and in the case of a lipstick, an amount of 2 to 15% by mass of the total cosmetic is ideal. Accordingly, in one aspect, the sum of the amounts of the component (A), the component (B), the component (C) and the additives permitted as cosmetics must not exceed 100% by mass but may be 100% by mass. In another aspect, the sum of the amounts of the component (A), the component (B) and the component (C) must not exceed 100% by mass but may be 100% by mass.

Any of the powders typically used in cosmetics may be used as the powder of the component (C). Specifically, extenders, color pigments and pearl pigments may be used.

Examples of the extenders include inorganic pigments such as silicic acid, silicic anhydride, magnesium silicate, talc, sericite, mica, kaolin, clay, bentonite, bismuth oxychloride, zirconium oxide, magnesium oxide, zinc oxide, aluminum oxide, calcium sulfate, barium sulfate, magnesium sulfate, calcium carbonate and magnesium carbonate, or composite powders of these pigments; organic powders such as polyamide, polyester, polypropylene, polystyrene, polyurethane, nylon, silicone resins, vinyl resins, urea resins, phenol resins, silicon resins, acrylic resins, melamine resins, epoxy resins, polycarbonate resins, divinylbenzene-styrene copolymers, silk powder, cellulose, N-ε-lauroyl-L-lysine, metal salts of long-chain alkyl phosphates, N-mono-long-chain alkyl acyl basic amino acids, and metal soaps, or composites of these powders; and composite powders of the above inorganic powders and the above organic powders. The particulate shape of these powders may be spherical, plate-like, needle-like, granular, or amorphous or the like.

In the present description, the expression “X, Y and/or Z” has the same meaning as “at least one selected from the group consisting of X, Y and Z”.

Examples of the color pigments include metal oxides such as titanium oxide, zinc oxide, yellow iron oxide, red iron oxide, bengala, black iron oxide, Prussian blue, ultramarine, chromium oxide and chromium hydroxide, or mixtures of these metal oxides; metal complexes such as manganese violet and cobalt titanate, or mixtures of such complexes; inorganic pigments such as carbon black; organic pigments such as tar dyes and lake pigments, or mixtures thereof; and natural colorants such as carmine or mixtures thereof.

Examples of pearl pigments that may be used include pearl pigments in which mica or synthetic phlogopite or the like has been coated with a colorant such as titanium oxide, iron oxide, silicon oxide, Prussian blue, chromium oxide, carmine, or an organic pigment or the like. These powders may also be subjected to any of various surface treatments prior to use using known methods, such as a water repellency treatment or a water and oil repellency treatment.

By including a powder having an ultraviolet-shielding effect such as a metal oxide powder as the powder of the component (C), a sunscreen cosmetic (cosmetic for preventing sunburn) can also be produced. If the metal oxide powder with an ultraviolet-shielding effect has an average particle size of 10 to 100 nm, then the generation of white powder residue upon application to the skin can be suppressed, which is particularly desirable. In those cases where the cosmetic according to the present invention is a sunscreen cosmetic, the amount of the liquid oily composition according to the present invention within the sunscreen cosmetic (namely, the proportion of the combination of the component (A) and the component (B)), relative to the total mass of the sunscreen cosmetic, is preferably within a range from 1 to 60% by mass, and the amount of the powder of the component (C) (here, the powder of the component (C) may be a powder having an ultraviolet-shielding effect such as a metal oxide powder), relative to the total mass of the sunscreen cosmetic, is preferably within a range from 5 to 30% by mass. The cosmetic according to the present invention can also be formed as a sunscreen cosmetic by including an organic ultraviolet absorber. Accordingly, in one aspect, the sum of the amounts of the component (A), the component (B), the component (C) (here, the powder of the component (C) may be a powder having an ultraviolet-shielding effect such as a metal oxide powder) and the additives permitted as cosmetics must not exceed 100% by mass but may be 100% by mass. In another aspect, the sum of the amounts of the component (A), the component (B) and the component (C) (here, the powder of the component (C) may be a powder having an ultraviolet-shielding effect such as a metal oxide powder) must not exceed 100% by mass but may be 100% by mass.

The cosmetic according to the present invention may also contain a volatile oil as a component (D), either instead of the component (C) and/or the additives permitted as cosmetics, or in addition to the component (C) and/or the additives permitted as cosmetics. In other words, one aspect of the cosmetic of the present invention contains the component (A), the component (B), the component (D), and one or more additives permitted as cosmetics, another aspect contains the component (A), the component (B), the component (C), the component (D), and one or more additives permitted as cosmetics, yet another aspect contains the component (A), the component (B), and the component (D), and yet another aspect contains the component (A), the component (B), the component (C), and the component (D).

A volatile oil is an oil that exhibits high volatility at normal temperatures, and specifically, describes an oil having a boiling point of 250° C. or lower. The volatile oil of the component (D) is a volatile hydrocarbon and/or a volatile silicone, as such oils exhibit favorable compatibility with the component (A) and the component (B). Examples of volatile oils that can be used favorably include cyclotetrasiloxane (also known as octamethylcyclotetrasiloxane), octamethyltrisiloxane, cyclopentasiloxane (also known as decamethylcyclopentasiloxane), decamethyltetrasiloxane, cyclohexasiloxane (also known as dodecamethylcyclohexasiloxane), dodecamethylpentasiloxane, light isoparaffin or light liquid isoparaffin, and isododecane.

The amount of the volatile oil, relative to the total mass of the cosmetic, is preferably within a range from 0.1 to 50% by mass, and the amount of the liquid oily composition described above (namely, the proportion of the combination of the component (A) and the component (B)) is preferably within a range from 1 to 99.9% by mass, and more preferably from 50 to 99.9% by mass. Further, in those cases where the cosmetic also includes the component (C), the amount of the component (C) is preferably within a range from 2 to 98% by mass of the total cosmetic. Further, the amount of the component (C) may be set to a more appropriate level depending on the type of cosmetic, and for example, in the case of a powder foundation, rouge or eye shadow, an amount of 70 to 98% by mass of the total cosmetic is ideal, in the case of a water-in-oil emulsion foundation, emulsion-type eye shadow or sunscreen, an amount of 5 to 30% by mass of the total cosmetic is ideal, and in the case of a lipstick, an amount of 2 to 15% by mass of the total cosmetic is ideal.

Accordingly, in one aspect, the sum of the amounts of the component (A), the component (B), the component (D) and the additives permitted as cosmetics must not exceed 100% by mass but may be 100% by mass. In another aspect, the sum of the amounts of the component (A), the component (B), the component (C), the component (D) and the additives permitted as cosmetics must not exceed 100% by mass but may be 100% by mass. Further, in another aspect, the sum of the amounts of the component (A), the component (B) and the component (D) must not exceed 100% by mass but may be 100% by mass. Furthermore, in yet another aspect, the sum of the amounts of the component (A), the component (B), the component (C) and the component (D) must not exceed 100% by mass but may be 100% by mass.

The cosmetic according to the present invention may also contain water as a component (E) and a surfactant as a component (F), either instead of the component (C), the component (D) and/or the additives permitted as cosmetics, or in addition to the component (C), the component (D) and/or the additives permitted as cosmetics. In other words, one aspect of the cosmetic of the present invention contains the component (A), the component (B), the component (E), the component (F), and one or more additives permitted as cosmetics, another aspect contains the component (A), the component (B), the component (C), the component (E), the component (F), and one or more additives permitted as cosmetics, yet another aspect contains the component (A), the component (B), the component (D), the component (E), the component (F), and one or more additives permitted as cosmetics, yet another aspect contains the component (A), the component (B), the component (C), the component (D), the component (E), the component (F), and one or more additives permitted as cosmetics, yet another aspect contains the component (A), the component (B), the component (E), and the component (F), yet another aspect contains the component (A), the component (B), the component (C), the component (E), and the component (F), yet another aspect contains the component (A), the component (B), the component (D), the component (E), and the component (F), and yet another aspect contains the component (A), the component (B), the component (C), the component (D), the component (E), and the component (F).

By including these components, the cosmetic according to the present invention can be formed as an emulsion cosmetic. The cosmetic according to the present invention may be either an oil-in-water emulsion cosmetic (O/W emulsion cosmetic) or a water-in-oil emulsion cosmetic (W/O emulsion cosmetic). In the case of a water-in-oil emulsion cosmetic, the favorable persistence of the cosmetic film and the pigment dispersibility effect that are features of the liquid oily composition according to the present invention manifest readily.

Examples of the types of water that may be used as the water of the component (E) include ion-exchanged water, distilled water, fruit-derived water, and deep-sea water. There are no particular limitations on the blend amount of the component (E), and for example, amounts within a range from 5 to 90% by mass relative to the total mass of the cosmetic can be used favorably.

Any surfactant typically able to be used in cosmetic products may be used as the surfactant of the component (F). Of the various possibilities, nonionic surfactants such as polyoxyethylene-modified silicones (also known as polyether-modified silicones), polyglycerol fatty acid esters, alkyl polyoxyethylenes, polyoxyethylene fatty acid esters, and polyoxyethylene hydrogenated castor oil are preferred.

Specific examples of the surfactant of the component (F) include polyoxyethylene (100) monostearate, glyceryl (SE) monostearate, polyoxyethylene (30) stearyl ether, sorbitan sesquioleate, and sorbitan polyoxyethylene (20 mol) monooleate.

There are no particular limitations on the blend amount of the component (F), and for example, amounts within a range from 0.01 to 10% by mass relative to the total mass of the cosmetic can be used favorably.

In the case of an oil-in-water emulsion cosmetic, the amount of the liquid oily composition according to the present invention, relative to the total mass of the cosmetic, is preferably within a range from 1 to 60% by mass, the amount of the surfactant (the component (F)) is preferably within a range from 0.01 to 5% by mass, and the amount of the aqueous component (the component (E)) is preferably within a range from 20 to 90% by mass. Moreover, by also adding a powder (the component (C)), an oil-in-water emulsion foundation or an oil-in-water emulsion sunscreen or the like can also be prepared.

In the case of a water-in-oil emulsion cosmetic, the amount of the liquid oily composition according to the present invention, relative to the total mass of the cosmetic, is preferably within a range from 1 to 60% by mass, the amount of the surfactant (the component (F)) is preferably within a range from 0.1 to 10% by mass, and the amount of the aqueous component (the component (E)) is preferably within a range from 5 to 70% by mass. Moreover, by also adding a powder (the component (C)), a water-in-oil emulsion foundation or a water-in-oil emulsion sunscreen or the like can also be prepared.

Accordingly, in one aspect, the sum of the amounts of the component (A), the component (B), the component (E), the component (F), and the additives permitted as cosmetics must not exceed 100% by mass but may be 100% by mass. In another aspect, the sum of the amounts of the component (A), the component (B), the component (C), the component (E), the component (F), and the additives permitted as cosmetics must not exceed 100% by mass but may be 100% by mass. Further, in another aspect, the sum of the amounts of the component (A), the component (B), the component (D), the component (E), the component (F), and the additives permitted as cosmetics must not exceed 100% by mass but may be 100% by mass. Furthermore, in yet another aspect, the sum of the amounts of the component (A), the component (B), the component (C), the component (D), the component (E), the component (F), and the additives permitted as cosmetics must not exceed 100% by mass but may be 100% by mass. In yet another aspect, the sum of the amounts of the component (A), the component (B), the component (E), and the component (F) must not exceed 100% by mass but may be 100% by mass. Further, in yet another aspect, the sum of the amounts of the component (A), the component (B), the component (C), the component (E), and the component (F) must not exceed 100% by mass but may be 100% by mass. Furthermore, in yet another aspect, the sum of the amounts of the component (A), the component (B), the component (D), the component (E), and the component (F) must not exceed 100% by mass but may be 100% by mass. Moreover, in yet another aspect, the sum of the amounts of the component (A), the component (B), the component (C), the component (D), the component (E), and the component (F) must not exceed 100% by mass but may be 100% by mass.

The cosmetic according to the present invention may also include the types of additives typically added to cosmetics, namely additives permitted as cosmetics, provided the effects of the present invention are not impaired. Examples of these types of additives include oily components (but excluding the component (A) and the component (B)), antioxidants, antioxidant assistants, preservatives, ultraviolet absorbers, monohydric alcohols, polyhydric alcohols, water-soluble polymers, pH regulators, inorganic salts or salts of organic acids, chelating agents, vitamins, organic solvents, fragrances, various extracts, and ultraviolet scattering agents. One of these additives maybe used alone, or a combination of two or more additives may be used. In this description, in those cases where one of the above additives is used in the cosmetic according to the present invention, if the additive functions as one of the components (A), (B), (C), (D) or (F) described above, then the additive is deemed to be the component (A), (B), (C), (D) or (F), and is not considered to represent an additive.

The oily components refer to oily components other than the component (A) and the component (B), and examples include hydrocarbons, fatty acid esters, triglycerides, fatty acids, higher alcohols, silicone resins, fluorine-based oils, and derivatives of these compounds. Specific examples include castor oil, olive oil, avocado oil, palm oil, cacao oil, liquid paraffin, liquid branched paraffin, vaseline, squalane, hydrogenated polyisobutene, hydrogenated polydecene, propanediol di(caprylate/caprate), neopentyl glycol dicaprate, triethylhexanoin (also known as tri-2-ethylhexanoic acid), butyl stearate, octyldodecyl myristate, isopropyl myristate, isopropyl lanolin fatty acid ester, hexyl lanolin fatty acid ester, diisopropyl adipate, diisopropyl sebacate, isotridecyl isononanoate, polyglyceryl decaisostearate, 2-octyldodecanol, dipentaerythritol fatty acid ester, di(phytosteryl/octyldodecyl) lauroyl glutamate, beeswax, dimer dilinoleyl bis(behenyl/isostearyl/phytosteryl) dimer dilinoleate, di(cholesteryl/octyldodecyl)N-lauroyl-L-glutamate, cetyl 2-ethylhexanoate, and oleyl alcohol.

Examples of the antioxidants include oil-soluble vitamin C derivatives, tocopherols and derivatives and salts thereof, dibutylhydroxytoluene, butylhydroxyanisole, and gallate esters. A single antioxidant may be used alone, or a combination of two or more antioxidants may be used.

Examples of the antioxidant assistants include phosphoric acid, citric acid, maleic acid, malonic acid, succinic acid, fumaric acid, cephalin, hexametaphosphate, phytic acid, and ethylenediaminetetraacetic acid. A single antioxidant assistant may be used alone, or a combination of two or more antioxidant assistants may be used.

Examples of the preservatives include methylparaben, ethylparaben, butylparaben and phenoxyethanol. A single preservative may be used alone, or a combination of two or more preservatives may be used.

Examples of the ultraviolet absorbers include benzoic acid-based ultraviolet absorbers such as para-aminobenzoic acid (hereinafter abbreviated as PABA), PABA monoglycerol ester, N,N-dipropoxy PABA ethyl ester, N,N-diethoxy PABA ethyl ester, N,N-dimethyl PABA ethyl ester, N,N-dimethyl PABA butyl ester, and N,N-dimethyl PABA ethyl ester; anthranilic acid-based ultraviolet absorbers such as homomenthyl-N-acetylanthranilate; salicylic acid-based ultraviolet absorbers such as amyl salicylate, menthyl salicylate, homomenthyl salicylate, octyl salicylate, phenyl salicylate, benzyl salicylate, and p-isopropanol phenyl salicylate; cinnamic acid-based ultraviolet absorbers such as octyl cinnamate, ethyl-4-isopropyl cinnamate, methyl-2,5-diisopropyl cinnamate, ethyl-2,4-diisopropyl cinnamate, methyl-2,4-diisopropyl cinnamate, propyl-p-methoxy cinnamate, isopropyl-p-methoxy cinnamate, isoamyl-p-methoxy cinnamate, octyl-p-methoxy cinnamate (2-ethylhexyl-p-methoxy cinnamate), 2-ethoxyethyl-p-methoxy cinnamate, cyclohexyl-p-methoxy cinnamate, ethyl-α-cyano-β-phenyl cinnamate, 2-ethylhexyl-α-cyano-β-phenyl cinnamate and glycerylmono-2-ethylhexanoyl-diparamethoxy cinnamate; benzophenone-based ultraviolet absorbers such as 2,4-dihydroxybenzophenone, 2,2′-dihydroxy-4-methoxybenzophenone, 2.2′-dihydroxy-4,4-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl-4′-phenylbenzophenone-2-carboxylate, 2-hydroxy-4-n-octoxybenzophenone, and 4-hydroxy-3-carboxybenzophenone; as well as 3-(4′-methylbenzylidene)-d,l-camphor, 3-benzylidene-d,l-camphor, urocanic acid, ethyl urocanate, 2-phenyl-5-methylbenzoxazole, 2,2′-hydroxy-5-methylphenylbenzotriazole, 2-(2′-hydroxy-5′-t-octylphenyl)benzotriazole, 2-(2′-hydroxy-5′-methylphenyl)benzotriazole, dibenzalazine, dianisoylmethane, 4-methoxy-4′-t butyldibenzoylmethane, 5-(3,3-dimethyl-2-norbornylidene)-3-pentan-2-one, 2,4,6-trianilino-p-(carbo-2′-ethylhexyl-1′-oxy)-1,3,5-triazine, 4-tert-butyl-4′-methoxydibenzoylmethane, hexyl ethylaminohydroxybenzoyl benzoate, ethylhexyltriazine, t-butylmethoxybenzoylmethane, bisethylhexyloxyphenol methoxyphenyltriazine, diethylhexylbutamidotriazone, and oxybenzone-3. A single ultraviolet absorber may be used alone, or a combination of two or more ultraviolet absorbers may be used.

The monohydric alcohol may be either a lower alcohol or a higher alcohol. Examples of the lower alcohols include methanol, ethanol, propanol, isopropanol, isobutyl alcohol, and t-butyl alcohol. Further, examples of the higher alcohols include cetanol (also known as cetyl alcohol and palmityl alcohol), stearyl alcohol (also known as octadecyl alcohol), isostearyl alcohol (also known as isooctadecanol), oleyl alcohol, cetostearyl alcohol, octyldodecanol, decyltetradecanol, hexyldecanol, behenyl alcohol, lauryl alcohol, lanolin alcohol, and hydrogenated lanolin alcohol.

Examples of the polyhydric alcohols include propylene glycol (1,2-propanediol), 1,3-propanediol, 1,3-butylene glycol (1,3-butanediol), pentylene glycol (1,2-pentanediol), neopentylene glycol (2,2-dimethyl-1,3-propanediol), isoprene glycol (3-methyl-1,3-butanediol), dipropylene glycol, glycerol, polyglycerol, polyethylene glycol, pentaerythritol, dipentaerythritol, sorbitol, and sorbitan. A single polyhydric alcohol may be used alone, or a combination of two or more polyhydric alcohols may be used.

The water-soluble polymer may be a natural water-soluble polymer, a semi-synthetic water-soluble polymer, or a synthetic water-soluble polymer. The water-soluble thickener included in the cosmetic according to the present invention may be either a single compound or a combination of two or more compounds.

Examples of the natural water-soluble polymers include plant-based polymers such as agar, glucomannan, gum arabic, tragacanth gum, galactan, guar gum, carob gum, karaya gum, carrageenan, pectin, quince seeds (marmelo-derived component), algae colloid (also known as algae extract), and starch (for example, starch from rice, corn, potato or wheat); microorganism-derived polymers such as xanthan gum, dextran, succinoglucan, and pullulan; and animal-based polymers such as collagen, casein, albumin, and gelatin.

Examples of the semi-synthetic water-soluble polymers include starch-based polymer such as carboxymethyl starch and methylhydroxypropyl starch; cellulose-based polymers such as methyl cellulose, nitrocellulose, methyl hydroxypropyl cellulose, sodium cellulose sulphate, hydroxypropyl cellulose, carboxymethyl cellulose, sodium carboxymethyl cellulose, crystalline cellulose, and cellulose powder; and alginic acid-based polymers such as sodium alginate and propylene glycol alginate.

Examples of the synthetic water-soluble polymers include vinyl-based polymers such as polyvinyl alcohol, polyvinyl methyl ether, polyvinylpyrrolidone, and carboxy vinyl polymer; polyoxyethylene-based polymers such as polyethylene glycol 20,000, 40,000 and 60,000; polyoxyethylene polyoxypropylene copolymer-based polymers; acrylic-based polymers such as sodium polyacrylate, polyethyl acrylate and polyacrylamide; as well as polyethyleneimine and cationic polymers.

Examples of the pH regulators include edetic acid, disodium edetate, citric acid, sodium citrate, sodium hydroxide, potassium hydroxide, and triethanolamine. A single pH regulator may be used alone, or a combination of two or more pH regulators may be used.

Examples of the inorganic salts include sodium chloride, potassium chloride, magnesium chloride, sodium sulphate, potassium sulphate, and magnesium sulphate. Examples of the salts of organic acids include citric acid, malic acid, tartaric acid and the salts thereof, ascorbic acid and the salts thereof, and ascorbic acid derivatives and the salts thereof.

Examples of the chelating agents include disodium edetate, edetic acid salts, and hydroxyethanediphosphonic acid. A single chelating agent may be used alone, or a combination of two or more chelating agents may be used.

Examples of the vitamins include vitamin A, vitamin B₁, vitamin B₂, vitamin B₆, vitamin E, vitamin K, derivatives of these vitamins, pantothenic acid and derivatives thereof, and biotin.

Examples of the extracts include plant extracts such as aloe vera, witch hazel, hamamelis, cucumber, lemon, lavender, and rose.

The cosmetic described above can be produced by mixing the liquid oily composition as required with the component (C), the component (D), the component (E), the component (F), and/or the additives permitted as cosmetics, and then either molding the mixture into a form appropriate for the desired cosmetic, or using the mixture to fill a container appropriate for the desired cosmetic. The mixing may be conducted by placing all the components in a mixing device simultaneously, or by conducting mixing while each component is added in an appropriate sequence. The stirring or kneading conducted during the mixing, or the molding into a form appropriate for the desired cosmetic or the filling of a container may use methods well known in the technical field. The temperature during the mixing may be set so that the temperature inside the mixer is within a range from 20 to 100° ° C.

In the production of the cosmetic, rather than using the liquid oily composition, the component (A) and the component (B) may be used prior to their use in the preparation of the liquid oily composition, but using the liquid oily composition in a form that has already been prepared as a cosmetic production raw material yields better production efficiency for the cosmetic. When the component (A) and the component (B) are used separately, the blend amounts of the component (A) and the component (B) within the total mass of the cosmetic may be set to achieve the same amounts as when the liquid oily composition is used.

One aspect of the present invention relates to the use, as a cosmetic, of the component (A), the component (B), and if desired the component (C), the component (D), the component (E), the component (F) and/or one or more additives permitted as cosmetics. Further, another aspect relates to the use, as a cosmetic, of the liquid oily composition, and if desired the component (C), the component (D), the component (E), the component (F) and/or one or more additives permitted as cosmetics.

Use as a cosmetic may be achieved using known methods in accordance with the purpose and form of the cosmetic. In other words, one aspect of the present invention is a cosmetic method that includes applying the cosmetic according to the present invention to an appropriate target region that requires makeup, such as the skin or hair. More specifically, for example, an oil-in-water moisturizing cream may be applied to a skin or hair moisturizing method that includes applying the cream to a target that requires moisturizing in an amount that yields a moisturizing effect. In a further example, an oil-in-water hand cream may be applied to a method for moisturizing the hands and/or preventing or improving rough hands, wherein the method includes applying the hand cream to the target hands that require moisturizing and/or prevention or improvement of rough hands, in an amount that yields a moisturizing effect and/or a prevention or improvement effect on rough hands. For example, a water-in-oil emulsion foundation or a powder foundation may be applied to a method for conditioning the surface state of skin or the skin color, wherein the method includes applying the foundation to target skin that requires conditioning of the surface state of the skin or the skin color, preferably skin on the face or around the neck or the like, in an amount that yields a conditioning effect on the surface state of the skin or the skin color. For example, a water-in-oil emulsion sunscreen may be applied to a method for preventing sunburn that includes applying the sunscreen to target skin that requires sunburn prevention in an amount that yields a sunburn prevention effect.

In one aspect, the cosmetic of the present invention contains from 3 to 18% by mass, and preferably from 5 to 15% by mass of the liquid oily composition, relative to the total mass of the cosmetic. In another aspect, the cosmetic of the present invention contains from 0.75 to 4.5% by mass of the component (A) and from 2.25 to 13.5% by mass of the component (B), and preferably from 1.25 to 3.75% by mass of the component (A) and from 3.75 to 11.25% by mass of the component (B), relative to the total mass of the cosmetic. Here, the component (A) is polyglyceryl-2 tri(2-(1,3,3-trimethylbutyl)-5,7,7-trimethyloctanoate) or di(2-(1,3,3-trimethylbutyl)-5,7,7-trimethyloctyl) malate, and the component (B) is dimethicone 10 cs.

In one aspect, the liquid oily composition of the present invention has a light feeling, an excellent water repellency effect, and excellent pigment dispersibility.

In one aspect, the cosmetic of the present invention has a light feeling, excellent persistence of the cosmetic film, and excellent storage stability, whereas in another aspect, the cosmetic of the present invention has a light feeling, excellent persistence of the cosmetic film, excellent storage stability, favorable coloring properties and minimal dulling, and in yet another aspect, the cosmetic of the present invention has a light feeling, excellent persistence of the cosmetic film, excellent storage stability, minimal dulling, and excellent water resistance.

### EXAMPLES

The present invention is described below in further detail based on a series of specific examples. However, the present invention is not limited in any way by the content of the examples described below.

First is an example of a liquid oily composition that represents one embodiment of the present invention.

Using the blend formulations shown in Tables 1 to 8, components were stirred and mixed under heating at 70° C. and then cooled to 25° C. to form a series of liquid oily compositions. Each obtained liquid oily composition was evaluated for water repellency, lightness of feelings, and pigment dispersibility. The evaluation results are shown in Tables 1 to 8. The numerical values shown in the component cells of the tables represent % by mass values. Further, in the tables, “(a)” indicates ester oils and “(b)” indicates silicone oils. The components (a) include ester oils corresponding with the component (A) according to the present invention. The components (b) include silicone oils corresponding with the component (B) according to the present invention. In the tables, “(a)/(b)” indicates the mass ratio between the component (a) and the component (b) (namely, the (a)/(b) ratio) in each sample.

### <Liquid Oily Composition Raw Materials>

For the raw materials of the liquid oily compositions shown in Tables 1 to 8, “COSMOL 43N (hydroxyl value: 40 mgKOH/g) manufactured by The Nisshin OilliO Group, Ltd. was used for the polyglyceryl-2 tri(2-(1,3,3-trimethylbutyl)-5,7,7-trimethyloctanoate), “SALACOS 3318” (hydroxyl value: 20 mgKOH/g) manufactured by The Nisshin OilliO Group, Ltd. was used for the glyceryl tri(2-(1,3,3-trimethylbutyl)-5,7,7-trimethyloctanoate), “COSMOL 222” (hydroxyl value: 80 mgKOH/g) manufactured by The Nisshin OilliO Group, Ltd. was used for the di(2-(1,3,3-trimethylbutyl)-5,7,7-trimethyloctyl) malate, and “SALACOS DP-518N” (hydroxyl value: 30 mgKOH/g) manufactured by The Nisshin OilliO Group, Ltd. was used as the dipentaerythrityl penta(2-(1,3,3-trimethylbutyl)-5,7,7-trimethyloctanoate). For the comparative examples, SALACOS 99″ (hydroxyl value: 0 mgKOH/g) manufactured by The Nisshin OilliO Group, Ltd. was used for the 3,5,5-trimethylhexyl 3,5,5-trimethylhexanoate, COSMOL 525″ (hydroxyl value: 0 mgKOH/g) manufactured by The Nisshin OilliO Group, Ltd. was used for the neopentyl glycol di-2-ethylhexanoate, and COSMOL 43V″ (hydroxyl value: 40 mgKOH/g) manufactured by The Nisshin OilliO Group, Ltd. was used for the polyglyceryl-2 triisostearate.

For the silicone oils, “KF-96A-5 cs” manufactured by Shin-Etsu Chemical Co., Ltd. was used for the dimethicone 5 cs, “KF-96A-10 cs” manufactured by Shin-Etsu Chemical Co., Ltd. was used for the dimethicone 10 cs, “KF-96A-30 cs” manufactured by Shin-Etsu Chemical Co., Ltd. was used for the dimethicone 30 cs, “KF-96A-3000 cs” manufactured by Shin-Etsu Chemical Co., Ltd. was used for the dimethicone 3,000 cs, “KF-96A-10000 cs” manufactured by Shin-Etsu Chemical Co., Ltd. was used for the dimethicone 10,000 cs, “KF-56A” manufactured by Shin-Etsu Chemical Co., Ltd. was used for the diphenylsiloxy phenyl trimethicone, and “KF-54” manufactured by Shin-Etsu Chemical Co., Ltd. was used for the diphenyl dimethicone.

### <Miscibility Evaluation Method>

The method used for evaluating the state when the components corresponding with the component (a) and the component (b) used in each of the examples and comparative examples were mixed in a mass ratio of 1:1, and the evaluation criteria are described below. The component (a) and the component (b) were weighed into the same mass screw-top vial, heated to 70° C., and then shaken to mix the components. Subsequently, the mass screw-top vial was left to stand overnight at 25° C., and the external appearance was then confirmed by visual inspection. The results are shown in Tables 1 to 8.

### (Miscibility Evaluation Criteria)

S: the external appearance was transparent and uniform.

IS: the external appearance revealed separation into two layers, turbidity, or cloudiness.

### <Water Repellency Evaluation Method>

The liquid oily composition of the evaluation target was applied to a 50 mm×50 mm PMMA plate, and excess oil fraction was wiped off with a waste cloth to achieve a coating amount within a range from 0.04 to 0.12 mg/cm². Subsequently, 30 μL of ion-exchanged water was dripped onto the coating film applied to the plate, the plate was gradually inclined, and the angle at which the water droplet began to move was measured.

### (Water Repellency Evaluation Criteria)

S: the droplet moved at a plate inclination angle of less than 18 degrees.

A: the droplet moved at a plate inclination angle of at least 18 degrees but less than 20 degrees.

B: the droplet moved at a plate inclination angle of at least 20 degrees but less than 23 degrees.

C: the droplet moved at a plate inclination angle of at least 23 degrees but less than 26 degrees.

D: the droplet moved at a plate inclination angle of at least 26 degrees but less than 29 degrees.

E: the droplet did not move even at a plate inclination angle of 29 degrees.

### <Evaluation Method for Lightness of Feeling>

First, 40 μL of the liquid oily composition of the evaluation target was dripped onto the inside of the forearm and massaged into the skin for 30 seconds using all the fingers of the opposite hand except for the thumb. Using glyceryl tri(caprylate/caprate), which is a known general-purpose ester oil with a light feeling, as a comparative ester oil, the lightness of the feeling of the liquid oily composition was evaluated by ten monitors.

### (Lightness of Feelings Evaluation Criteria)

S: 9 or more of the 10 monitors answered that the lightness of feeling was either similar or superior to that of the comparative ester oil.

A: 6 to 8 of the 10 monitors answered that the lightness of feeling was either similar or superior to that of the comparative ester oil.

B: 4 or 5 of the 10 monitors answered that the lightness of feeling was either similar or superior to that of the comparative ester oil.

C: 2 or 3 of the 10 monitors answered that the lightness of feeling was either similar or superior to that of the comparative ester oil.

D: 1 or fewer of the 10 monitors answered that the lightness of feeling was either similar or superior to that of the comparative ester oil.

### <Pigment Dispersibility Evaluation Method>

First, 0.1 g of titanium oxide with an average particle size of 0.15 μm (“TIPAQUE A-100” manufactured by Ishihara Sangyo Kaisha, Ltd.) and 10 g of the liquid oily composition produced in one of the blend formulations shown in Tables 1 to 8 were weighed into a glass screw-top vial, and the vial was sealed with a lid. The vial was placed in an ultrasonic cleaning device and subjected to an ultrasound treatment for 10 minutes with occasional shaking of the vial. One hour after the ultrasound treatment, a fluorescent light was shone through the screw-top vial from the rear, and the external appearance was evaluated based on the proportion of the height of the shaded portion in which the dispersed pigment reduced the transmission of the light relative to a value of 100% for the total height of the mixed liquid of the pigment and the liquid oily composition.

### (Pigment Dispersibility Evaluation Criteria)

S: at least 90% of the height was shaded.

A: at least 80% but less than 90% of the height was shaded.

B: at least 70% but less than 80% of the height was shaded.

C: at least 50% but less than 70% of the height was shaded.

D: at least 30% but less than 50% of the height was shaded.

E: less than 30% of the height was shaded.

### <Overall Evaluation of Water Repellency, Lightness of Feeling, and Pigment Dispersibility>

For each of the liquid oily compositions of the evaluation targets, the evaluation scores for the water repellency, the lightness of feeling and the pigment dispersibility were numericalized with S representing 16 points, A representing 8 points, B representing 4 points, C representing 2 points, D representing 1 point and E representing 0 points, and the combined score for the evaluations of water repellency, lightness of feeling and pigment dispersibility was recorded as the overall evaluation score.

TABLE 1

| | | Exam- | Exam- | Exam- | Exam- |
|---|---|---|---|---|---|
| | | ple | ple | ple | ple |
| | Component | 1 | 2 | 3 | 4 |
| (a) | polyglyceryl-2 tri(2- | 25 | — | — | — |
| | (1,3,3-trimethylbutyl)- | | | | |
| | 5,7,7-trimethyloctanoate) | | | | |
| | glyceryl tri(2-(1,3,3- | — | 25 | — | — |
| | trimethylbutyl)-5,7,7- | | | | |
| | trimethyloctanoate) | | | | |
| | di(2-(1,3,3- | — | — | 25 | — |
| | trimethylbutyl)-5,7,7- | | | | |
| | trimethyloctyl) malate | | | | |
| | dipentaerythrityl | — | — | — | 25 |
| | penta(2-(1,3,3- | | | | |
| | trimethylbutyl)- | | | | |
| | 5,7,7-trimethyloctanoate) | | | | |
| (b) | dimethicone 10 cs | 75 | 75 | 75 | 75 |

| | | | | |
|---|---|---|---|---|
| External appearance (S: good | S | S | S | S |
| miscibility, transparent, | | | | |
| IS: poor miscibility, separation) | | | | |
| Miscibility when (a):(b) = 1:1 | S | S | S | S |
| (a)/(b) | 0.33 | 0.33 | 0.33 | 0.33 |
| Water repellency | S | A | A | B |
| Lightness of feeling | S | S | S | S |
| Pigment dispersibility | S | B | S | B |
| Overall evaluation score | 48 | 28 | 40 | 24 |

TABLE 2

| | | Comparative | Comparative | Comparative | Comparative | Comparative |
|---|---|---|---|---|---|---|
| | Component | Example 1 | Example 2 | Example 3 | Example 4 | Example 5 |
| (a) | polyglyceryl-2 tri(2-(1,3,3-trimethylbutyl)- | 100 | — | — | — | — |
| | 5,7,7-trimethyloctanoate) | | | | | |
| | glyceryl tri(2-(1,3,3-trimethylbutyl)-5,7,7- | — | 100 | — | — | — |
| | trimethyloctanoate) | | | | | |
| | di(2-(1,3,3-trimethylbutyl)-5,7,7- | — | — | 100 | — | — |
| | trimethyloctyl) malate | | | | | |
| | dipentaerythrityl penta(2-(1,3,3- | — | — | — | 100 | — |
| | trimethylbutyl)-5,7,7-trimethyloctanoate) | | | | | |
| (b) | dimethicone 10 cs | — | — | — | — | 100 |

| | | | | | |
|---|---|---|---|---|---|
| Miscibility when (a):(b) = 1:1 | — | — | — | — | — |
| (a)/(b) | 0.33 | 0.33 | 0.33 | 0.33 | 0.33 |
| Water repellency | E | E | E | E | C |
| Lightness of feeling | D | D | D | D | S |
| Pigment dispersibility | S | A | S | S | E |
| Overall evaluation score | 17 | 9 | 17 | 17 | 18 |

TABLE 3

| | | Comparative | Comparative | Comparative | Comparative | Comparative | Comparative |
|---|---|---|---|---|---|---|---|
| | Component | Example 6 | Example 7 | Example 8 | Example 9 | Example 10 | Example 11 |
| (a) | 3,5,5-trimethylhexyl 3,5,5- | 25 | 25 | — | — | — | — |
| | trimethylhexanoate | | | | | | |
| | neopentyl glycol di-2- | — | — | 25 | 25 | — | — |
| | ethylhexanoate | | | | | | |
| | polyglyceryl-2 triisostearate | — | — | — | — | 25 | 25 |
| (b) | dimethicone 10 cs | 75 | — | 75 | — | 75 | — |
| | dimethicone 10,000 cs | — | 75 | — | 75 | — | 75 |

| | | | | | | |
|---|---|---|---|---|---|---|
| External appearance (S: good miscibility, | S | S | S | S | IS | IS |
| transparent, IS: poor miscibility, separation) | | | | | | |
| Miscibility when (a):(b) = 1:1 | S | S | S | S | IS | IS |
| (a)/(b) | 0.33 | 0.33 | 0.33 | 0.33 | 0.33 | 0.33 |
| Water repellency | C | C | C | C | D | D |
| Lightness of feeling | S | D | S | D | C | C |
| Pigment dispersibility | E | E | E | E | E | E |
| Overall evaluation score | 18 | 3 | 18 | 3 | 3 | 3 |

TABLE 4

| | | Exam- | Exam- | Exam- | Exam- |
|---|---|---|---|---|---|
| | | ple | ple | ple | ple |
| | Component | 5 | 6 | 7 | 8 |
| (a) | polyglyceryl-2 tri(2- | 25 | 25 | — | — |
| | (1,3,3-trimethylbutyl)- | | | | |
| | 5,7,7-trimethyloctanoate) | | | | |
| | di(2-(1,3,3- | — | — | 25 | 25 |
| | trimethylbutyl)-5,7,7- | | | | |
| | trimethyloctyl) malate | | | | |
| (b) | dimethicone 1 cs | 75 | — | 75 | — |
| | dimethicone 5 cs | — | 75 | — | 75 |

| | | | | |
|---|---|---|---|---|
| External appearance (S: good | S | S | S | S |
| miscibility, transparent, | | | | |
| IS: poor miscibility, separation) | | | | |
| Miscibility when (a):(b) = 1:1 | S | S | S | S |
| (a)/(b) | 0.33 | 0.33 | 0.33 | 0.33 |
| Water repellency | B | A | B | A |
| Lightness of feeling | S | S | S | S |
| Pigment dispersibility | S | S | S | S |
| Overall evaluation score | 36 | 40 | 36 | 40 |

TABLE 5

| | | Exam- | Exam- | Exam- | Exam- |
|---|---|---|---|---|---|
| | | ple | ple | ple | ple |
| Table 4 | Component | 9 | 10 | 11 | 12 |
| (a) | polyglyceryl-2 tri(2- | 25 | 25 | — | — |
| | (1,3,3-trimethylbutyl)- | | | | |
| | 5,7,7-trimethyloctanoate) | | | | |
| | di(2-(1,3,3- | — | — | 25 | 25 |
| | trimethylbutyl)-5,7,7- | | | | |
| | trimethyloctyl) malate | | | | |
| (b) | diphenylsiloxy | 75 | — | 75 | — |
| | phenyl trimethicone | | | | |
| | diphenyl dimethicone | — | 75 | — | 75 |

| | | | | |
|---|---|---|---|---|
| External appearance (S: good | S | S | S | S |
| miscibility, transparent, | | | | |
| IS: poor miscibility, separation) | | | | |
| Miscibility when (a):(b) = 1:1 | S | S | S | S |
| (a)/(b) | 0.33 | 0.33 | 0.33 | 0.33 |
| Water repellency | S | S | A | A |
| Lightness of feeling | S | S | S | S |
| Pigment dispersibility | S | S | S | S |
| Overall evaluation score | 48 | 48 | 40 | 40 |

TABLE 6

| | | Comparative | Comparative | Comparative | Comparative | Comparative | Comparative |
|---|---|---|---|---|---|---|---|
| | Component | Example 12 | Example 13 | Example 14 | Example 15 | Example 16 | Example 17 |
| (a) | polyglyceryl-2 tri(2-(1,3,3- | 25 | 25 | 25 | — | — | — |
| | trimethylbutyl)-5,7,7- | | | | | | |
| | trimethyloctanoate) | | | | | | |
| | di(2-(1,3,3-trimethylbutyl)-5,7,7- | — | — | — | 25 | 25 | 25 |
| | trimethyloctyl) malate | | | | | | |
| (b) | dimethicone 30 cs | 75 | — | — | 75 | — | — |
| | dimethicone 3,000 cs | — | 75 | — | — | 75 | — |
| | dimethicone 10,000 cs | = | — | 75 | — | — | 75 |

| | | | | | | |
|---|---|---|---|---|---|---|
| External appearance (S: good miscibility, | IS | IS | IS | IS | IS | IS |
| transparent, IS: poor miscibility, separation) | | | | | | |
| Miscibility when (a):(b) = 1:1 | IS | IS | IS | IS | IS | IS |
| (a)/(b) | 0.33 | 0.33 | 0.33 | 0.33 | 0.33 | 0.33 |
| Water repellency | C | C | C | C | C | C |
| Lightness of feeling | C | D | D | C | D | D |
| Pigment dispersibility | E | C | E | E | C | E |
| Overall evaluation score | 5 | 19 | 3 | 5 | 19 | 3 |

As illustrated in Tables 1 to 6, the compositions of the present invention from Examples 1 to 12, which contained a combination of an ester oil having a 2-(1,3,3-trimethylbutyl)-5,7,7-trimethyloctyl structure in the aliphatic chain, having a hydroxyl value within a range from 5 to 100 mgKOH/g, and being liquid at 20° C., with a silicone oil that was miscible with that ester oil in a mass ratio of 1:1 and is liquid at 20° C., were confirmed as having an excellent water repellency effect, a light feeling, and excellent pigment dispersibility. In contrast, none of the compositions of Comparative Examples 1 to 5, which were compositions that lacked one of the component (a) and the component (b) of the present invention, were able to satisfy all of the conditions of a superior water repellency effect, a light feeling, and excellent pigment dispersibility. Among the various results, of particular note was that the compositions corresponding with the examples of the present invention exhibited even better water repellency than the composition of Comparative Example 5, which evaluated a lone silicone oil conventionally known to have an excellent water repellency effect. Further, in the case of the compositions of Comparative Examples 6 to 9, although the ester oil was an ester oil that is liquid at 20° C. and is conventionally known to exhibit good miscibility with silicone compounds, the structure of the aliphatic chain differs from the ester oil used as the component (A) in the present invention, and the range for the hydroxyl value is also different. In these comparative examples, the pigment dispersibility was particularly poor, and the water repellency was also inadequate. Moreover, in Comparative Examples 10 and 11, although the ester oil of the component (a) exhibited a range for the hydroxyl value and an alcohol skeleton for the ester that were similar to the ester oil of the component (a) used in Example 1, the structure of the aliphatic chain was different. In the compositions of these comparative examples, not only was the pigment dispersibility poor, but water repellency and the lightness of the feeling were also unsatisfactory. Comparative Examples 12 to 17 represent examples in which polyglyceryl-2 tri(2-(1,3,3-trimethylbutyl)-5,7,7-trimethyloctanoate) or di(2-(1,3,3-trimethylbutyl)-5,7,7-trimethyloctyl) malate was used as the component (a), and a silicone oil that was not miscible with the component (a) in a mass ratio of 1:1 was used as the component (b), and in each case, the water repellency, the lightness of the feeling, and the pigment dispersibility were all inferior.

TABLE 7

| | | Exam- | Exam- | Exam- | Exam- | Exam- | Exam- | Comparative | Comparative |
|---|---|---|---|---|---|---|---|---|---|
| | Component | ple 13 | ple 14 | ple 15 | ple 16 | ple 17 | ple 18 | Example 18 | Example 19 |
| (a) | polyglyceryl-2 tri(2-(1,3,3- | 5 | 10 | 30 | 50 | 75 | 80 | 2 | 90 |
| | trimethylbutyl)-5,7,7- | | | | | | | | |
| | trimethyloctanoate) | | | | | | | | |
| (b) | dimethicone 10 cs | 95 | 90 | 70 | 50 | 25 | 20 | 98 | 10 |

| | | | | | | | | |
|---|---|---|---|---|---|---|---|---|
| External appearance (S: good miscibility, | S | S | S | S | S | S | S | S |
| transparent, IS: poor miscibility, separation) | | | | | | | | |
| Miscibility when (a):(b) = 1:1 | S | S | S | S | S | S | S | S |
| (a)/(b) | 0.05 | 0.11 | 0.43 | 1 | 3 | 4 | 0.02 | 9 |
| Water repellency | A | S | A | A | A | A | C | E |
| Lightness of feeling | S | S | S | B | B | B | S | D |
| Pigment dispersibility | B | B | S | S | A | A | D | A |
| Overall evaluation score | 28 | 36 | 40 | 28 | 20 | 20 | 19 | 9 |

TABLE 8

| | | Exam- | Exam- | Exam- | Exam- | Exam- | Exam- | Comparative | Comparative |
|---|---|---|---|---|---|---|---|---|---|
| | Component | ple 19 | ple 20 | ple 21 | ple 22 | ple 23 | ple 24 | Example 20 | Example 21 |
| (a) | di(2-(1,3,3-trimethylbutyl)- | 5 | 10 | 30 | 50 | 75 | 80 | 2 | 90 |
| | 5,7,7-trimethyloctyl) malate | | | | | | | | |
| (b) | dimethicone 10 cs | 95 | 90 | 70 | 50 | 25 | 20 | 98 | 10 |

| | | | | | | | | |
|---|---|---|---|---|---|---|---|---|
| External appearance (S: good miscibility, | S | S | S | S | S | S | S | S |
| transparent, IS: poor miscibility, separation) | | | | | | | | |
| Miscibility when (a):(b) = 1:1 | S | S | S | S | S | S | S | S |
| (a)/(b) | 0.05 | 0.11 | 0.43 | 1 | 3 | 4 | 0.02 | 9 |
| Water repellency | B | A | B | B | A | A | C | E |
| Lightness of feeling | S | S | S | B | B | B | S | D |
| Pigment dispersibility | B | B | S | S | A | A | D | A |
| Overall evaluation score | 24 | 28 | 36 | 24 | 20 | 20 | 19 | 9 |

Moreover, as shown in Examples 13 to 24 in Tables 7 and 8, it was confirmed that compositions in which the (a)/(b) ratio that was the ratio between the component (a) and the component (b) was altered within a range from 0.05 to 4 exhibited an excellent water repellency effect, a light feeling, and excellent pigment dispersibility. Among these examples, the compositions of Examples 1, 15 and 21 in which the (a)/(b) ratio indicating the ratio between the component (a) and the component (b) was 0.33 or 0.43 exhibited particularly superior effects.

Next is an example of a cosmetic containing the liquid oily composition that represents another embodiment of the present invention. The cosmetics of Examples 25 to 34 and Comparative Examples 22 to 31 shown in Tables 9 to 13 were prepared using the methods described following each of the tables and were then evaluated using the evaluation methods and evaluation criteria described below. For all the cosmetics, evaluations were conducted for “lightness of feeling”, “persistence of the cosmetic film” and “storage stability”. In the case of foundations, additional evaluations were conducted for “coloring properties” and “dulling”, and for the sunscreens, additional evaluations were conducted for “dulling” and “water resistance”.

### <Evaluation Method for Lightness of Feeling>

The cosmetic of the evaluation target was applied to the inside of the forearm, and the feeling was evaluated. Using a cosmetic containing glyceryl tri(caprylate/caprate) as a standard sample for comparing the feeling, the lightness of the feeling of the cosmetic was evaluated by ten monitors by comparison with the standard sample.

### (Lightness of Feeling Evaluation Criteria)

S: 9 or more of the 10 monitors answered that the lightness of feeling was either similar or superior to that of the standard sample.

A: 6 to 8 of the 10 monitors answered that the lightness of feeling was either similar or superior to that of the standard sample.

B: 4 or 5 of the 10 monitors answered that the lightness of feeling was either similar or superior to that of the standard sample.

C: 2 or 3 of the 10 monitors answered that the lightness of feeling was either similar or superior to that of the standard sample.

D: 1 or fewer of the 10 monitors answered that the lightness of feeling was either similar or superior to that of the standard sample.

### <Evaluation Method for Persistence of Cosmetic Film>

The cosmetic of the evaluation target was first applied to the inside of the forearm (application (a)). One hour after the application, the same cosmetic was applied to a new location on the inside of the forearm and left to stand for 10 minutes (application (b)). The difference in the feeling between the locations of the application (a) and the application (b) was evaluated by 10 monitors.

### (Persistence of Cosmetic Film Evaluation Criteria)

S: 8 or more of the 10 monitors answered that there was little difference between the feeling of the application (a) and the application (b).

A: 6 or 7 of the 10 monitors answered that there was little difference between the feeling of the application (a) and the application (b).

B: 4 or 5 of the 10 monitors answered that there was little difference between the feeling of the application (a) and the application (b).

C: 2 or 3 of the 10 monitors answered that there was little difference between the feeling of the application (a) and the application (b).

D: 1 or fewer of the 10 monitors answered that there was little difference between the feeling of the application (a) and the application (b).

### <Evaluation Method for Storage Stability>

The cosmetic of the evaluation target was placed in a glass screw-top vial, the vial was sealed, and following storage for 14 days while the temperature was changed alternately between 40° ° C. (12 hours) and 5° C. (12 hours), the external appearance of the cosmetic was evaluated.

### (Storage Stability Evaluation Criteria)

A: almost no separation or settling was confirmed.

B: slight separation and/or settling was confirmed.

C: definite separation and/or settling was confirmed.

D: marked separation and/or settling was confirmed.

### <Evaluation Method for Coloring Properties>

The foundation of the evaluation target was applied to the inside of the forearm, and the coloring properties were evaluated. The coloring properties were evaluated by 10 monitors by comparing the color of the cosmetic and the color upon application to the skin and confirming any difference in the hue.

### (Coloring Properties Evaluation Criteria)

S: 9 or more of the 10 monitors answered that there was no difference or almost no difference between the colors.

A: 6 to 8 of the 10 monitors answered that there was no difference or almost no difference between the colors.

B: 4 or 5 of the 10 monitors answered that there was no difference or almost no difference between the colors.

C: 2 or 3 of the 10 monitors answered that there was no difference or almost no difference between the colors.

D: 1 or fewer of the 10 monitors answered that there was no difference or almost no difference between the colors.

### <Evaluation Method for Dulling>

The foundation or sunscreen cosmetic of the evaluation target was applied to the inside of the forearm (application (a)). One hour after the application, the same cosmetic was applied to a new location on the inside of the forearm and left to stand for 10 minutes (application (b)). The difference in the external appearance of the locations of the application (a) and the application (b) was evaluated by 10 monitors.

### (Dulling Evaluation Criteria)

S: 8 or more of the 10 monitors answered that there was little difference in the external appearance of the application (a) and the application (b).

A: 6 or 7 of the 10 monitors answered that there was little difference in the external appearance of the application (a) and the application (b).

B: 4 or 5 of the 10 monitors answered that there was little difference in the external appearance of the application (a) and the application (b).

C: 2 or 3 of the 10 monitors answered that there was little difference in the external appearance of the application (a) and the application (b).

D: 1 or fewer of the 10 monitors answered that there was little difference in the external appearance of the application (a) and the application (b).

### <Evaluation Method for Water Resistance>

The sunscreen cosmetic of the evaluation target was applied to a 50 mm×50 mm PMMA plate (unevenness: 5 μm) in an amount of 1.3 mg/cm² and was spread out to form a uniform coating. After standing for at least 15 minutes at room temperature to dry, the SPF was measured using a UV-1000 manufactured by Labsphere, Inc. (measurement (c)). The PMMA plate was secured at an inclination of 45 degrees and then washed with running tap water for one minute by pouring tap water at a rate of 150 mL per minute and from a height of 40 cm onto the top edge of the sample-coated surface of the plate.

Following this running water washing, the plate was left to dry for at least 15 minutes at room temperature, and the SPF was then measured again (measurement (d)). Using the formula shown below, the degree of persistence of the SPF effect was calculated. Here, if the SPF degree of persistence is simply calculated as [measured value of measurement (d)]/[measured value of measurement (c)], then if the measured value of the measurement (c) is low from the start, then even if the measured value of the measurement (d) is low, the residual ratio may appear to be high in some cases, even though the level of the SPF effect is inadequate for practical purposes. Accordingly, in order to also reflect the absolute value of the measured value of the measurement (d) following the washing with running water, a formula was used in which the value of [measured value of measurement (d)]/[measured value of measurement (c)] was multiplied by the measured value of measurement (d).

$$[ SPF degree of persistence ] = measured value of measurement ( d ) \times [ measured value of measurement ( d ) ] \text{} / [ measured value of measurement ( c ) ]$$

### (Water Resistance Evaluation Criteria)

S: SPF degree of persistence was 3.0 or higher.

A: SPF degree of persistence was at least 2.5 but less than 3.0.

B: SPF degree of persistence was at least 2.0 but less than 2.5.

C: SPF degree of persistence was at least 1.5 but less than 2.0.

D: SPF degree of persistence was less than 1.5.

### [Oil-in-Water Emulsion Moisturizing Cream]

An oil-in-water moisturizing cream was produced using the liquid oily composition of Example 1 as the liquid oily composition according to the present invention, namely a composition prepared by mixing polyglyceryl-2 tri(2-(1,3,3-trimethylbutyl)-5,7,7-trimethyloctanoate) (hydroxyl value: 40 mgKOH/g) as the component (A) and dimethicone 10 cs as the component (B) in an (A)/(B) ratio of 0.33. The formulation and the evaluation results for this oil-in-water moisturizing cream are shown in Table 9. The product “O.D.O” manufactured by The Nisshin OilliO Group, Ltd. can be used as glyceryl tri(caprylate/caprate).

The oil-in-water emulsion moisturizing cream was produced using the following steps A to C.

A: Components 1 to 12 were dissolved by heating and mixed uniformly.

B: Components 13 to 18 were heated and mixed uniformly.

C: At a temperature of 80° C., the mixture obtained in step A was added to the mixture obtained in step B and emulsified, and following cooling, the component 19 was added.

TABLE 9
Oil-in-water emulsion moisturizing cream

| | | | | | | Reference |
|---|---|---|---|---|---|---|
| | | | | Comparative | Comparative | Comparative |
| | | Example 25 | Example 26 | Example 22 | Example 23 | Example 1 |
| Components (raw materials) | | % by mass | % by mass | % by mass | % by mass | % by mass |
| 1 | liquid oily composition of Example 1 | 6.0 | — | — | — | — |
| 2 | polyglyceryl-2 tri(2-(1,3,3- | — | 1.5 | 6.0 | — | — |
| | trimethylbutyl)-5,7,7-trimethyloctanoate) | | | | | |
| 3 | dimethicone 10 cs | — | 4.5 | — | 6.0 | — |
| 4 | glyceryl tri(caprylate/caprate) | — | — | — | — | 6.0 |
| 5 | squalane | 2.0 | 2.0 | 2.0 | 2.0 | 2.0 |
| 6 | glyceryl tri-2-ethylhexanoate | 2.0 | 2.0 | 2.0 | 2.0 | 2.0 |
| 7 | dipentaerythritol fatty acid ester | 1.0 | 1.0 | 1.0 | 1.0 | 1.0 |
| 8 | di(phytosteryl/octyldodecyl) lauroyl | 1.0 | 1.0 | 1.0 | 1.0 | 1.0 |
| | glutamate | | | | | |
| 9 | cetanol | 2.0 | 2.0 | 2.0 | 2.0 | 2.0 |
| 10 | beeswax | 1.0 | 1.0 | 1.0 | 1.0 | 1.0 |
| 11 | polyoxyethylene (100) monostearate | 0.8 | 0.8 | 0.8 | 0.8 | 0.8 |
| 12 | glyceryl monostearate (SE) | 0.2 | 0.2 | 0.2 | 0.2 | 0.2 |
| 13 | glycerol | 5.0 | 5.0 | 5.0 | 5.0 | 5.0 |
| 14 | 1,3-butylene glycol | 10.0 | 10.0 | 10.0 | 10.0 | 10.0 |
| 15 | sodium hydroxide | 0.05 | 0.05 | 0.05 | 0.05 | 0.05 |
| 16 | methyl paraoxybenzoate | 0.2 | 0.2 | 0.2 | 0.2 | 0.2 |
| 17 | carboxy vinyl polymer | 0.2 | 0.2 | 0.2 | 0.2 | 0.2 |
| 18 | ion-exchanged water | 68.45 | 68.45 | 68.45 | 68.45 | 68.45 |
| 19 | fragrance | 0.1 | 0.1 | 0.1 | 0.1 | 0.1 |

| | | | | | |
|---|---|---|---|---|---|
| Total | 100.0 | 100.0 | 100.0 | 100.0 | 100.0 |
| Lightness of feeling | S | S | D | S | — |
| Persistence of cosmetic film | S | S | B | D | |
| Storage stability | A | A | B | D | |

In the evaluations of the lightness of feeling of the oil-in-water emulsion moisturizing creams of Examples 25 and 26 and Comparative Examples 22 and 23, the oil-in-water emulsion moisturizing cream of Reference Comparative Example 1 was used as a standard sample. As illustrated in Table 9, the oil-in-water emulsion moisturizing cream of Example 25 that contained the liquid oily composition of Example 1, and the oil-in-water emulsion moisturizing cream of Example 26 that contained the component (A) and the component (B) added separately exhibited a light feeling, displayed favorable persistence of the cosmetic film, and also had favorable storage stability. In contrast, the oil-in-water emulsion moisturizing creams of Comparative Examples 22 and 23 which lacked either the component (A) or the component (B) that represent the essential components of the liquid oily composition according to the present invention were unable to satisfy all of the requirements of a light feeling, persistence of the cosmetic film and storage stability.

### [Oil-in-Water Emulsion Hand Cream]

An oil-in-water emulsion hand cream was produced using the liquid oily composition of Example 3 as an oily moisturizer, namely a composition prepared by mixing di(2-(1,3,3-trimethylbutyl)-5,7,7-trimethyloctyl) malate (hydroxyl value: 80 mgKOH/g) as the component (A) and dimethicone 10 cs as the component (B) in an (A)/(B) ratio of 0.33. The formulation and the evaluation results for this oil-in-water emulsion hand cream are shown in Table 10.

The oil-in-water emulsion hand cream was produced using the following steps A to C.

A: Components 1 to 10 were dissolved by heating and mixed uniformly.

B: Components 11 to 15 were heated and mixed uniformly.

C: At a temperature of 80° C., the mixture obtained in step A was added to the mixture obtained in step B and emulsified, and the resulting emulsion was then cooled.

TABLE 10
Water-in-oil emulsion hand cream

| | | | | | | Reference |
|---|---|---|---|---|---|---|
| | | | | Comparative | Comparative | Comparative |
| | | Example 27 | Example 28 | Example 24 | Example 25 | Example 2 |
| Components (raw materials) | | % by mass | % by mass | % by mass | % by mass | % by mass |
| 1 | liquid oily composition of Example 3 | 15.0 | — | — | — | — |
| 2 | di(2-(1,3,3-trimethylbutyl)-5,7,7- | — | 3.75 | 15.0 | — | — |
| | trimethyloctyl) malate | | | | | |
| 3 | dimethicone 10 cs | — | 11.25 | — | 15.0 | — |
| 4 | glyceryl tri(caprylate/caprate) | — | — | — | — | 15.0 |
| 5 | dimer dilinoleyl bis(behenyl/isostearyl/ | 1.0 | 1.0 | 1.0 | 1.0 | 1.0 |
| | phytosteryl) dimer dilinoleate | | | | | |
| 6 | di(phytosteryl/octyldodecyl) lauroyl | 1.0 | 1.0 | 1.0 | 1.0 | 1.0 |
| | glutamate | | | | | |
| 7 | cetanol | 2.0 | 2.0 | 2.0 | 2.0 | 2.0 |
| 8 | polyoxyethylene (30) stearyl ether | 0.5 | 0.5 | 0.5 | 0.5 | 0.5 |
| 9 | polyoxyethylene (100) monostearate | 2.0 | 2.0 | 2.0 | 2.0 | 2.0 |
| 10 | glyceryl monostearate (SE) | 2.0 | 2.0 | 2.0 | 2.0 | 2.0 |
| 11 | glycerol | 5.0 | 5.0 | 5.0 | 5.0 | 5.0 |
| 12 | 1,3-butylene glycol | 5.0 | 5.0 | 5.0 | 5.0 | 5.0 |
| 13 | phenoxyethanol | 0.5 | 0.5 | 0.5 | 0.5 | 0.5 |
| 14 | xanthan gum | 0.2 | 0.2 | 0.2 | 0.2 | 0.2 |
| 15 | ion-exchanged water | 65.8 | 65.8 | 65.8 | 65.8 | 65.8 |

| | | | | | |
|---|---|---|---|---|---|
| Total | 100.0 | 100.0 | 100.0 | 100.0 | 100.0 |
| Lightness of feeling | S | S | D | S | — |
| Persistence of cosmetic film | S | S | B | D | |
| Storage stability | A | A | A | D | |

In the evaluations of the lightness of feeling of the oil-in-water emulsion hand creams of Examples 27 and 28 and Comparative Examples 24 and 25, the oil-in-water emulsion hand cream of Reference Comparative Example 2 was used as a standard sample. As illustrated in Table 10, the oil-in-water emulsion hand cream of Example 27 that contained the liquid oily composition of Example 3, and the oil-in-water emulsion hand cream of Example 28 that contained the component (A) and the component (B) added separately exhibited a light feeling, displayed favorable persistence of the cosmetic film, and also had favorable storage stability. In contrast, the oil-in-water emulsion hand creams of Comparative Examples 24 and 25 which lacked either the component (A) or the component (B) that represent the essential components of the liquid oily composition according to the present invention were unable to satisfy all of the requirements of a light feeling, persistence of the cosmetic film and storage stability.

### [Water-In-Oil Emulsion Foundation]

A water-in-oil emulsion foundation was produced using the liquid oily composition of Example 1 as the liquid oily composition, namely a composition prepared by mixing polyglyceryl-2 tri(2-(1,3,3-trimethylbutyl)-5,7,7-trimethyloctanoate) (hydroxyl value: 40 mgKOH/g) as the component (A) and dimethicone 10 cs as the component (B) in an (A)/(B) ratio of 0.33. The formulation and the evaluation results for this water-in-oil emulsion foundation are shown in Table 11. The product “KSG-16” manufactured by Shin-Etsu Chemical Co., Ltd. can be used as the mixture of (dimethicone/vinyl dimethicone) cross polymer and cyclopentasiloxane, the product “KSP-100” manufactured by Shin-Etsu Chemical Co., Ltd. can be used as the (vinyl dimethicone/methicone silsesquioxane) cross polymer, the product “KF-6017” manufactured by Shin-Etsu Chemical Co., Ltd. can be used as the polyether-modified silicone, and the product “BENTONE 38” manufactured by Elementis plc can be used as the organic-modified clay mineral.

The water-in-oil emulsion foundation was produced using the following steps A to C.

A: Components 1 to 12 were dissolved by heating, and following cooling to 40° ° C., components 13 to 22 were added and dispersed using a homo mixer.

B: Components 23 to 28 were mixed uniformly and dissolved.

C: The mixture obtained in step B was added to the dispersion obtained in step A and emulsified.

TABLE 11
Water-in-oil emulsion foundation

| | | | | | | Reference |
|---|---|---|---|---|---|---|
| | | | | Comparative | Comparative | Comparative |
| | | Example 29 | Example 30 | Example 26 | Example 27 | Example 3 |
| Components (raw materials) | | % by mass | % by mass | % by mass | % by mass | % by mass |
| 1 | liquid oily composition of Example 1 | 5.0 | — | — | — | — |
| 2 | polyglyceryl-2 tri(2-(1,3,3- | — | 1.25 | 5.0 | — | — |
| | trimethylbutyl)-5,7,7-trimethyloctanoate) | | | | | |
| 3 | dimethicone 10 cs | — | 3.75 | — | 5.0 | — |
| 4 | glyceryl tri(caprylate/caprate) | — | — | — | — | 5.0 |
| 5 | octamethylcyclotetrasiloxane | 20.5 | 20.5 | 20.5 | 20.5 | 20.5 |
| 6 | (dimethicone/vinyl dimethicone) cross | 1.0 | 1.0 | 1.0 | 1.0 | 1.0 |
| | polymer, cyclopentasiloxane | | | | | |
| 7 | (vinyl dimethicone/methicone | 1.0 | 1.0 | 1.0 | 1.0 | 1.0 |
| | silsesquioxane) cross polymer | | | | | |
| 8 | squalane | 1.0 | 1.0 | 1.0 | 1.0 | 1.0 |
| 9 | di(cholesteryl•octyldodecyl) N-lauroyl-L- | 1.0 | 1.0 | 1.0 | 1.0 | 1.0 |
| | glutamate | | | | | |
| 10 | cetyl 2-ethylhexanoate | 1.0 | 1.0 | 1.0 | 1.0 | 1.0 |
| 11 | polyether-modified silicone | 0.5 | 0.5 | 0.5 | 0.5 | 0.5 |
| 12 | Sorbitan sesquioleate | 0.5 | 0.5 | 0.5 | 0.5 | 0.5 |
| 13 | organic-modified clay mineral | 0.5 | 0.5 | 0.5 | 0.5 | 0.5 |
| 14 | titanium oxide | 7.0 | 7.0 | 7.0 | 7.0 | 7.0 |
| 15 | zinc oxide | 3.0 | 3.0 | 3.0 | 3.0 | 3.0 |
| 16 | talc | 4.7 | 4.7 | 4.7 | 4.7 | 4.7 |
| 17 | mica | 2.0 | 2.0 | 2.0 | 2.0 | 2.0 |
| 18 | red iron oxide | 0.2 | 0.2 | 0.2 | 0.2 | 0.2 |
| 19 | yellow iron oxide | 1.6 | 1.6 | 1.6 | 1.6 | 1.6 |
| 20 | black iron oxide | 0.2 | 0.2 | 0.2 | 0.2 | 0.2 |
| 21 | nylon | 2.0 | 2.0 | 2.0 | 2.0 | 2.0 |
| 22 | titanated mica | 2.0 | 2.0 | 2.0 | 2.0 | 2.0 |
| 23 | purified water | 35.0 | 35.0 | 35.0 | 35.0 | 35.0 |
| 24 | ethanol | 5.0 | 5.0 | 5.0 | 5.0 | 5.0 |
| 25 | glycerol | 5.0 | 5.0 | 5.0 | 5.0 | 5.0 |
| 26 | antioxidant (dl-α-tocopherol) | 0.1 | 0.1 | 0.1 | 0.1 | 0.1 |
| 27 | hyaluronic acid | 0.1 | 0.1 | 0.1 | 0.1 | 0.1 |
| 28 | fragrance | 0.1 | 0.1 | 0.1 | 0.1 | 0.1 |

| | | | | | |
|---|---|---|---|---|---|
| Total | 100.0 | 100.0 | 100.0 | 100.0 | 100.0 |
| Lightness of feeling | S | S | C | S | — |
| Persistence of cosmetic film | S | S | D | D | |
| Storage stability | A | A | A | D | |
| Coloring properties | S | S | A | D | |
| Dulling | S | S | C | C | |

In the evaluations of the lightness of feeling of the water-in-oil emulsion foundations of Examples 29 and 30 and Comparative Examples 26 and 27, the water-in-oil emulsion foundation of Reference Comparative Example 3 was used as a standard sample. As illustrated in Table 11, the water-in-oil emulsion foundation of Example 29 that contained the liquid oily composition of Example 1, and the water-in-oil emulsion foundation of Example 30 that contained the component (A) and the component (B) added separately exhibited a light feeling, displayed favorable persistence of the cosmetic film, had favorable storage stability, exhibited excellent coloring properties, and suffered from little dulling. In contrast, the water-in-oil emulsion foundations of Comparative Examples 26 and 27 which lacked either the component (A) or the component (B) that represent the essential components of the liquid oily composition according to the present invention were unable to satisfy all of the requirements of a light feeling, persistence of the cosmetic film, storage stability, coloring properties, and dulling.

### [Powder Foundation]

A powder foundation was produced using the liquid oily composition of Example 1 as the liquid oily composition, namely a composition prepared by mixing polyglyceryl-2 tri(2-(1,3,3-trimethylbutyl)-5,7,7-trimethyloctanoate) (hydroxyl value: 40 mgKOH/g) as the component (A) and dimethicone 10 cs as the component (B) in an (A)/(B) ratio of 0.33. The formulation and the evaluation results for this powder foundation are shown in Table 12. The product “KMP-591” manufactured by Shin-Etsu Chemical Co., Ltd. can be used as the polymethylsilsesquioxane, and the product “KSP-100” manufactured by Shin-Etsu Chemical Co., Ltd. can be used as the (vinyl dimethicone/methicone silsesquioxane) cross polymer.

The powder foundation was produced using the following steps A to D.

A: Components 1 to 6 were heated to 50° ° C. and mixed.

B: Components 7 to 15 were mixed and dispersed.

C: The mixture obtained in step A was added to the mixed dispersion obtained in step B and mixed.

D: The mixture obtained in step C was powdered and compression-molded into a plate.

TABLE 12
Powder foundation

| | | | | | | Reference |
|---|---|---|---|---|---|---|
| | | | | Comparative | Comparative | Comparative |
| | | Example 31 | Example 32 | Example 28 | Example 29 | Example 4 |
| Components (raw materials) | | % by mass | % by mass | % by mass | % by mass | % by mass |
| 1 | liquid oily composition of Example 1 | 12.0 | — | — | — | — |
| 2 | polyglyceryl-2 tri(2-(1,3,3- | — | 3.0 | 12.0 | — | — |
| | trimethylbutyl)-5,7,7-trimethyloctanoate) | | | | | |
| 3 | dimethicone 10 cs | — | 9.0 | — | 12.0 | — |
| 4 | glyceryl tri(caprylate/caprate) | — | — | — | — | 12.0 |
| 5 | squalane | 5.0 | 5.0 | 5.0 | 5.0 | 5.0 |
| 6 | methyl paraoxybenzoate | 0.5 | 0.5 | 0.5 | 0.5 | 0.5 |
| 7 | titanium oxide | 5.0 | 5.0 | 5.0 | 5.0 | 5.0 |
| 8 | bengala | 0.5 | 0.5 | 0.5 | 0.5 | 0.5 |
| 9 | yellow iron oxide | 1.2 | 1.2 | 1.2 | 1.2 | 1.2 |
| 10 | black iron oxide | 0.1 | 0.1 | 0.1 | 0.1 | 0.1 |
| 11 | sericite | 50.0 | 50.0 | 50.0 | 50.0 | 50.0 |
| 12 | mica | 16.0 | 16.0 | 16.0 | 16.0 | 16.0 |
| 13 | talc | 5.7 | 5.7 | 5.7 | 5.7 | 5.7 |
| 14 | polymethylsilsesquioxane | 2.0 | 2.0 | 2.0 | 2.0 | 2.0 |
| 15 | (vinyl dimethicone/methicone | 2.0 | 2.0 | 2.0 | 2.0 | 2.0 |
| | silsesquioxane) cross polymer | | | | | |

| | | | | | |
|---|---|---|---|---|---|
| Total | 100.0 | 100.0 | 100.0 | 100.0 | 100.0 |
| Lightness of feeling | S | S | D | S | — |
| Persistence of cosmetic film | S | S | C | D | |
| Storage stability | A | A | A | D | |
| Coloring properties | S | S | A | D | |
| Dulling | S | S | C | C | |

In the evaluations of the lightness of feeling of the powder foundations of Examples 31 and 32 and Comparative Examples 28 and 29, the powder foundation of Reference Comparative Example 4 was used as a standard sample. As illustrated in Table 12, the powder foundation of Example 31 that contained the liquid oily composition of Example 1, and the powder foundation of Example 32 that contained the component (A) and the component (B) added separately exhibited a light feeling, displayed favorable persistence of the cosmetic film, had favorable storage stability, exhibited excellent coloring properties, and suffered from little dulling. In contrast, the powder foundations of Comparative Examples 28 and 29 which lacked either the component (A) or the component (B) that represent the essential components of the liquid oily composition according to the present invention were unable to satisfy all of the requirements of a light feeling, persistence of the cosmetic film, storage stability, coloring properties, and dulling.

### [Water-in-Oil Emulsion Sunscreen]

A water-in-oil emulsion foundation was produced using the liquid oily composition of Example 1 as the liquid oily composition, namely a composition prepared by mixing polyglyceryl-2 tri(2-(1,3,3-trimethylbutyl)-5,7,7-trimethyloctanoate) (hydroxyl value: 40 mgKOH/g) as the component (A) and dimethicone 10 cs as the component (B) in an (A)/(B) ratio of 0.33. The formulation and the evaluation results for this water-in-oil emulsion sunscreen are shown in Table 13. The product “TIPAQUE TTO-S2” manufactured by Ishihara Sangyo Kaisha, Ltd. can be used as the stearic acid-treated titanium oxide microparticles, the product “FINEX 25” manufactured by Sakai Chemical Industry Co., Ltd. treated with 5% of methylhydrogenpolysiloxane can be used as the silicone-treated zinc oxide, the product “KF-7312” manufactured by Shin-Etsu Chemical Co., Ltd. can be used as the mixture of trimethoxysiloxysilicate and cyclopentasiloxane, and the product “ABIL EM-90” manufactured by Evonik Industries AG can be used as the cetyl dimethicone copolyol.

The water-in-oil emulsion sunscreen was produced using the following steps A to D.

A: Components 1 to 13 were mixed uniformly.

B: Components 14 to 17 were mixed uniformly.

C: The mixture obtained in step B was added to the mixture obtained in step A and emulsified.

D: The emulsion obtained in step C was used to fill a resin bottle containing a stainless steel ball.

TABLE 13
Water-in-oil emulsion sunscreen

| | | | | | | Reference |
|---|---|---|---|---|---|---|
| | | | | Comparative | Comparative | Comparative |
| | | Example 33 | Example 34 | Example 30 | Example 31 | Example 5 |
| Components (raw materials) | | % by mass | % by mass | % by mass | % by mass | % by mass |
| 1 | liquid oily composition of Example 1 | 10.0 | — | — | — | — |
| 2 | polyglyceryl-2 tri(2-(1,3,3- | — | 2.5 | 10.0 | — | — |
| | trimethylbutyl)-5,7,7-trimethyloctanoate) | | | | | |
| 3 | dimethicone 10 cs | — | 7.5 | — | 10.0 | — |
| 4 | glyceryl tri(caprylate/caprate) | — | — | — | — | 10.0 |
| 5 | stearic acid-treated titanium oxide | 5.0 | 5.0 | 5.0 | 5.0 | 5.0 |
| | microparticles | | | | | |
| 6 | silicone-treated zinc oxide | 5.0 | 5.0 | 5.0 | 5.0 | 5.0 |
| 7 | 2-ethylhexyl para-methoxycinnamate | 5.0 | 5.0 | 5.0 | 5.0 | 5.0 |
| 8 | cyclopentadimethicone | 11.9 | 11.9 | 11.9 | 11.9 | 11.9 |
| 9 | trimethoxysiloxysilicate, | 2.0 | 2.0 | 2.0 | 2.0 | 2.0 |
| | cyclopentasiloxane | | | | | |
| 10 | cetyl dimethicone copolyol | 2.0 | 2.0 | 2.0 | 2.0 | 2.0 |
| 11 | sorbitan polyoxyethylene (20 mol) | 0.2 | 0.2 | 0.2 | 0.2 | 0.2 |
| | monooleate | | | | | |
| 12 | sorbitan sesquioleate | 0.8 | 0.8 | 0.8 | 0.8 | 0.8 |
| 13 | nylon powder | 2.0 | 2.0 | 2.0 | 2.0 | 2.0 |
| 14 | 1,3-butylene glycol | 5.0 | 5.0 | 5.0 | 5.0 | 5.0 |
| 15 | ethanol | 5.0 | 5.0 | 5.0 | 5.0 | 5.0 |
| 16 | phenoxyethanol | 0.5 | 0.5 | 0.5 | 0.5 | 0.5 |
| 17 | sodium chloride | 0.5 | 0.5 | 0.5 | 0.5 | 0.5 |
| 18 | lon-exchanged water | 45.0 | 45.0 | 45.0 | 45.0 | 45.0 |
| 19 | fragrance | 0.1 | 0.1 | 0.1 | 0.1 | 0.1 |

| | | | | | |
|---|---|---|---|---|---|
| Total | 100.0 | 100.0 | 100.0 | 100.0 | 100.0 |
| Lightness of feeling | S | S | D | S | — |
| Persistence of cosmetic film | S | S | C | D | |
| Storage stability | A | A | A | D | |
| Dulling | S | S | C | C | |
| Water resistance | S | S | B | C | |

In the evaluations of the lightness of feeling of the water-in-oil emulsion sunscreens of Examples 33 and 34 and Comparative Examples 30 and 31, the water-in-oil emulsion sunscreen of Reference Comparative Example 5 was used as a standard sample. As illustrated in Table 13, the water-in-oil emulsion sunscreen of Example 33 that contained the liquid oily composition of Example 1, and the water-in-oil emulsion sunscreen of Example 34 that contained the component (A) and the component (B) added separately exhibited a light feeling, displayed favorable persistence of the cosmetic film, had favorable storage stability, suffered from little dulling, and exhibited good water resistance. In contrast, the water-in-oil emulsion sunscreens of Comparative Examples 30 and 31 which lacked either the component (A) or the component (B) that represent the essential components of the liquid oily composition according to the present invention were unable to satisfy all of the requirements of a light feeling, persistence of the cosmetic film, storage stability, water resistance, and dulling.

### INDUSTRIAL APPLICABILITY

The present invention is able to provide a liquid oily composition that has a light feeling, an excellent water repellency effect or excellent pigment dispersibility, or is capable of imparting persistence to a cosmetic film and excellent storage stability when used as a base oil in any of various cosmetics, or this liquid oily composition is particularly suitable as a raw material for makeup cosmetics and sunscreen cosmetics and the like which require favorable persistence of the cosmetic film. Further, by including the liquid oily composition as one oily component, a cosmetic can be provided that exhibits favorable sensations upon use, with excellent persistence of the cosmetic film, favorable coloring properties and minimal dulling, or exhibiting excellent storage stability or enabling the imparting of water resistance.

1. A liquid oily composition comprising a component (A) and a component (B) described below, wherein
a mass ratio ((A)/(B)) of an amount of the component (A) with respect to the total composition, relative to an amount of the component (B) with respect to the total composition, is within a range from 0.05 to 4:
Component (A): an ester oil having a 2-(1,3,3-trimethylbutyl)-5,7,7-trimethyloctyl structure in the aliphatic chain, having a hydroxyl value within a range from 5 to 100 mgKOH/g, and being liquid at 20° C.,
Component (B): a silicone oil that is miscible with the component (A) in a mass ratio of 1:1 and is liquid at 20° C.

2. The liquid oily composition according to claim 1, wherein the component (A) is at least one compound selected from the group consisting of polyglyceryl-2 tri(2-(1,3,3-trimethylbutyl)-5,7,7-trimethyloctanoate), di(2-(1,3,3-trimethylbutyl)-5,7,7-trimethyloctyl) malate, glyceryl tri(2-(1,3,3-trimethylbutyl)-5,7,7-trimethyloctanoate), and dipentaerythrityl penta(2-(1,3,3-trimethylbutyl)-5,7,7-trimethyloctanoate).

3. The liquid oily composition according to claim 1, wherein the component (B) is at least one compound selected from the group consisting of dimethicone, diphenylsiloxy phenyl trimethicone, diphenyl dimethicone and phenyl trimethicone.

4. The liquid oily composition according to claim 1, wherein the component (A) is at least one compound selected from the group consisting of polyglyceryl-2 tri(2-(1,3,3-trimethylbutyl)-5,7,7-trimethyloctanoate), glyceryl tri(2-(1,3,3-trimethylbutyl)-5,7,7-trimethyloctanoate) and dipentaerythrityl penta(2-(1,3,3-trimethylbutyl)-5,7,7-trimethyloctanoate), and the component (B) is dimethicone.

5. The liquid oily composition according to claim 1, wherein the component (B) has a viscosity at 20° ° C. within a range from 2 to 300 mPa·s.

6. A cosmetic comprising the liquid oily composition according to claim 1.

7. The cosmetic according to claim 6, further comprising a component (C) described below:
Component (C): a powder.

8. The cosmetic according to claim 6, further comprising a component (D) described below:
Component (D): a volatile oil that is a volatile hydrocarbon and/or a volatile silicone.

9. The cosmetic according to claim 6, further comprising a component (E) and a component (F), and being an O/W emulsion cosmetic or a W/O emulsion cosmetic:
Component (E): water,
Component (F): a surfactant.

10. A cosmetic comprising a component (A) and a component (B) described below, wherein
a mass ratio ((A)/(B)) of an amount of the component (A) with respect to the total mass of the cosmetic, relative to an amount of the component (B) with respect to the total mass of the cosmetic, is within a range from 0.05 to 4:
Component (A): an ester oil having a 2-(1,3,3-trimethylbutyl)-5,7,7-trimethyloctyl structure in the aliphatic chain, having a hydroxyl value within a range from 5 to 100 mgKOH/g, and being liquid at 20° C.,
Component (B): a silicone oil that is miscible with the component (A) in a mass ratio of 1:1 and is liquid at 20° C.

11. The cosmetic according to claim 10, wherein the component (A) is at least one compound selected from the group consisting of polyglyceryl-2 tri(2-(1,3,3-trimethylbutyl)-5,7,7-trimethyloctanoate), di(2-(1,3,3-trimethylbutyl)-5,7,7-trimethyloctyl) malate, glyceryl tri(2-(1,3,3-trimethylbutyl)-5,7,7-trimethyloctanoate), and dipentaerythrityl penta(2-(1,3,3-trimethylbutyl)-5,7,7-trimethyloctanoate).

12. The cosmetic according to claim 10, wherein the component (B) is at least one compound selected from the group consisting of dimethicone, diphenylsiloxy phenyl trimethicone, diphenyl dimethicone and phenyl trimethicone.

13. The cosmetic according to claim 10, wherein the component (A) is at least one compound selected from the group consisting of polyglyceryl-2 tri(2-(1,3,3-trimethylbutyl)-5,7,7-trimethyloctanoate), glyceryl tri(2-(1,3,3-trimethylbutyl)-5,7,7-trimethyloctanoate), and dipentaerythrityl penta(2-(1,3,3-trimethylbutyl)-5,7,7-trimethyloctanoate), and
the component (B) is dimethicone.

14. The cosmetic according to claim 10, further comprising a component (C) described below:
Component (C): a powder.

15. The cosmetic according to claim 10, further comprising a component (D) described below:
Component (D): a volatile oil that is a volatile hydrocarbon and/or a volatile silicone.

16. The cosmetic according to claim 10, further comprising a component (E) and a component (F), and being an O/W emulsion cosmetic or a W/O emulsion cosmetic:
Component (E): water,
Component (F): a surfactant.

17. A method for imparting water resistance to a cosmetic, comprising adding effective amount of the liquid oily composition according to claim 1 to the cosmetic as a base oil thereof.
